(19) United States
(12) Patent Application Publication
(10) Pub. No.: US 2018/0293911 A1
(43) Pub. Date: Oct. 11, 2018

(54) SYSTEMS AND METHODS FOR TRACKING AND EXCHANGING WELLNESS, FITNESS, AND PRODUCTIVITY DATA USING AN ELECTRONIC PLATFORM

(71) Applicant: FitBliss, Inc., San Jose, CA (US)

(72) Inventor: Navid Rastegar-Panah, Saratoga, CA (US)

(21) Appl. No.: 15/949,839

(22) Filed: Apr. 10, 2018

Related U.S. Application Data
(60) Provisional application No. 62/483,693, filed on Apr. 10, 2017.

(51) Int. Cl. G09B 19/00; G06F 17/30; G06Q 10/06; G16H 20/30; G09B 5/02; A63B 24/00

(52) U.S. Cl. CPC G09B 19/00; G06F 17/30551; G06Q 10/06398; G16H 20/30; G06Q 30/01; G09B 19/0092; A63B 24/0062; G06Q 10/06393; G09B 5/02

(57) ABSTRACT

A method is described herein that comprises receiving from the user a selection of a fitness activity. The method includes receiving from the user a productivity goal for achieving a productivity objective, the receiving including indicating a time frame for achieving the at least one productivity goal. The method includes using historical data of the user to identify and recommend a fitness goal, wherein the historical data includes fitness activity data of the fitness activity previously performed by the user and productivity data of productivity objectives previously achieved by the user over a common time frame. The method includes correlating the fitness activity data and the productivity data to identify an interdependence between the fitness activity data and the productivity data, the using the historical data of the user comprising using information of the interdependence to identify and recommend the fitness goal.

### RELATED APPLICATIONS

This application claims the benefit of U.S. Application No. 62/483,693, filed Apr. 10, 2017

### TECHNICAL FIELD

The disclosure herein involves tracking and exchanging personal fitness and productivity metrics data using an electronic data platform.

### BACKGROUND

Companies lose an average of $500 Billion in lost productivity every year. Contributors to this loss include poor health, poor work culture, lack of engagement (only 32% of work/sales force are actively engaged in key enterprise tasks), and high stress.

Obesity is one of the leading causes of health issues that collectively costs the US healthcare system $150 Billion annually. Among Americans, $120 Million are overweight, 108 Million annually make multiple attempts to lose weight, and $20 Billion is spent annually on weight loss, yet less than 1% of those who attempt weight loss are successful. Existing treatments have had little impact on this epidemic due to poor efficacy and/or numerous safety issues.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### BRIEF DESCRIPTION OF THE DRAWINGS

FIG. 1 describes a schematic of classes and their relationship between each other under an embodiment.

FIG. 2 provides an example of FitProductivity workflow, under an embodiment.

FIG. 3 shows data collected with respect to FitGoals, under an embodiment.

FIG. 4 shows data collected with respect to FitSummary, under an embodiment.

FIG. 5 shows data collected with respect to FitStats, under an embodiment.

FIG. 6 shows data collected with respect to FitTips and FitTips Feedback, under an embodiment.

FIG. 7 shows data collected with respect to FitPrediction, under an embodiment.

FIG. 8 shows an introduction page, under an embodiment.

FIG. 9 shows an interface for setting a new goal, under an embodiment.

FIG. 10 shows an interface for setting a new goal, under an embodiment.

FIG. 11 shows a congratulations page, under an embodiment, under an embodiment.

FIG. 12 shows a FitGoals summary page, under an embodiment.

FIG. 13 shows an email sent to user summarizing progress with respect to a goal, under an embodiment.

FIG. 14 shows a FitTips page that summarizes tips either liked by a user or marked as completed by a user, under an embodiment.

FIG. 15 shows a summary page for a user, under an embodiment.

FIG. 16A shows step data over time, under an embodiment.

FIG. 16B shows step data over time, under an embodiment.

FIG. 17 shows exercise steps trend and daily steps target, under an embodiment.

FIG. 18 shows date, weight and steps goal by day of week, under an embodiment.

FIG. 19 shows weight gain/loss over time, under an embodiment.

FIG. 20 shows an autocorrelation function plot, under an embodiment.

FIG. 21 shows an partial autocorrelation function plot, under an embodiment.

FIG. 22A shows a of residual-fitted plot, under an embodiment.

FIG. 22B shows a residuals density plot, under an embodiment.

FIG. 23 shows predicted steps trend, under an embodiment.

FIG. 24 shows a fit tip, under an embodiment.

FIG. 25 shows a process of data collection and integration, under an embodiment.

FIG. 26 shows steps of data collection and integration, under an embodiment.

### DETAILED DESCRIPTION

A FitBliss platform is described herein for tracking fitness and wellness data. The FitBliss platform also provides a FitProductivity component which tracks performance metrics. The FitBliss platform allows a user to track fitness metrics against productivity metrics. Note that the terms FitBliss platform and FitProductivity platform may be used interchangeably to refer to an overall fitness and productivity metrics tracking application.

Employee may under an embodiment have access to the FitBliss platform within Salesforce.com and other tools like Workday™, FinancialForce.com, Fairsail™, and more. These other tools focus on Human Capital Management, where employees can track their onboarding, work performance, Paid Time Off tracking, and employee benefits.

FitBliss may use its native tracking system to bring in or incorporate Fitness & Wellness Key Performance Indicators (KPIs) like steps, distance, activity minutes, sleep, water intake, floors, and more.

FitBliss users may view their Customer Relationship Management (CRM) KPIs within Salesforce.com. These are leads created, call volume, opportunities created, tasks completed, opportunities closed, support cases closed, support case Net Promoter Scores (NPS), support case Customer Satisfaction Score (CSAT), campaigns created, total responses from campaigns, and more.

FitBliss provides 4 main features within FitProductivity:

FitStats

FitStats is a representation of your KPIs selected and the date ranges

FitPrediction

FitPrediction allows the employee to set a numerical productivity goal. FitPrediction then users the employee's own historical fitness and productivity data to provide a numerical fitness goal based on their selected CRM KPI, i.e. how much Fitness & Wellness KPI they would need to complete in order to achieve that CRM KPI. As one example, a user selects ‘Week’ for timeframe, ‘Leads Created’ for CRM KPI, and inputs a goal of 3 (leads created) on average daily for that week. User then selects the Fitness & Wellness KPI to see what they would need to achieve in order to hit their CRM KPI Goal they inputted (in this case 3). They would then click on Generate and the platform populates the Fitness and Wellness (FitBliss) KPI for the user to see. This data is strictly based on the employee's previous week's data.

FitGoals

FitGoals is a tool for the employee to save a personally identified CRM Goal attached to personally estimated output of fitness and wellness KPI. The Goal is time based with a start and end date for user to hit the goal. FitGoals provide daily updates based on the time frame.

FitTips

The platform provides 4 Tips a Day. Tips are tracked against a user's record. If a user hits ‘x’ number of ‘likes’ or ‘did it!’ buttons, the platform provides an email notification saying congrats, you've built a healthy behavior and asking the user if the user wants the same tips or new tips to keep the user engaged with a personalized experience. The platform provides context on the new tips if they want a new tip.

If a user sends an email past 6 PM local time, the platform sends an email about burnout the next morning. The emails sent out by the user are tracked within Salesforce. These emails are typically tracked with the Salesforce technology that tracks sales and service related activities; sending emails, making phone calls, etc. The personalized email is based on ‘FitConnect Activity’ or ‘Routine Log’ mood and time stamp. FitConnect Activity is an activity that is done using a synced fitness app or wearable that the user has logged in that system/technology. A Routine Log is a self-reported log of an activity inside the native FitBliss activity tracking system. Also, the personalized email may include a nutritional tip. FitTips may be integrated into the FitSummary/FitResults page which shows the user the progress the user has made including days they have mitigated a burnout episode, removed burnout, etc.

To start, the audience for the FitProductivity Suite may be Sales, Service, and Marketing Reps, under an embodiment.

The Goal of FitProductivity is to help find the optimal fitness/wellness levels for each rep individually, and then share the high level insights to the employer as Business Intelligence. The FitBliss platform may also share a ‘Forecast’ based on historical trends of their employees' fitness/wellness performance and the relationship between fitness/wellness performance and sales, service, and marketing KPIs.

FitProductivity's focus is to share insights on fitness levels against CRM KPIs (Leads Created, Opportunities Created, Opportunities Closed, Cases Closed, & Campaigns Created (i.e. a marketing campaign)). FitBliss shows historical Fitness KPI data of the sales, service, or marketing rep alongside their CRM KPIs.

Within FitProductivity, there are 3 main features under an embodiment:

FitGoals—This allows a user to enter a CRM KPI Goal, and FitBliss then pumps out a fitness level a user need to attain in order to achieve the CRM KPI Goal. The formula may be based on the users optimal CRM KPI against what their Fitness KPIs were during that same time frame, i.e. a user's optimal performance of CRM Leads created was 21 Leads in a week, averaging 3 leads a day (Sunday-Saturday). During that same period of time, user achieved 70,000 steps, averaging 10,000 steps per day. Now, the sales rep types the name of the Goal (i.e. Get 15 Leads!), then clicks on ‘Week’ in the timeframe, then selects ‘Leads Created’ as the Goal KPI, then types 15 leads for the goal the next 7 days (week). FitBliss would then suggest the number of daily steps, in this case we know that 1 lead at optimal is 3,333 steps, and the goal is to get to a daily step average that equals the daily lead creation number (15 divided by 7=2.14 leads per day), so it'll be 2.14*3,333 steps which equals 7,143 daily steps, the number of daily steps the user needs to achieve based on historical data. (The user may also type the user's own goal). Then the platform would ask if the user would like to get morning emails giving them insights on their progress against this goal. If they say yes, the platform provides them with daily emails for the next 7 days sharing their goal attainment. At the end of the FitGoal Duration (being 7 days since the user selected week time frame), the platform provides an email summary of their optimal day of steps (or another selected fitness KPI that FitBliss provides) vs their optimal lead created date throughout those 7 days.

FitTips—This is under on embodiment a small widget on the page where a user sees 4 standard tips based on the time of day between midnight and 8 am, 8:01 AM-noon, 12:01-5, 5:01-11:59 PM. The widget provides an image and a short tip. The goal here is to get the user to use our FitTips to contribute toward achievement of their FitGoal Fitness KPI, which then translates into better CRM KPIs. The platform then tracks which tips users “like” daily as tips are reset daily to make sure the user is doing the right things for themselves, i.e. creating a routine for instance of drinking 3 glasses of water before noon. When they click on ‘Like’ or ‘Did It!’ then we'll source that against the user's profile for machine learning and data mining for future ways to either recognize, engage, or educate the platform on more relevant tips to provide that user. We'll also know time of day the user typically engages with tips based on the timestamp of clicking on the tip. The platform under one embodiment adds a comment section or under another embodiment provides a ‘Share’ tip to Chatter Social Feed capability. These actions may then be tracked for analytics.

FitSummary or FitResults—This comprises dashboard type view for the user to see results. CRM & Fitness KPI averages (based on selecting Daily, Weekly, Monthly), FitTips marked ‘Did’, FitGoals achieved, Top 3 Activates Logged with a Fitness App or FitBliss Native Workout Tracker, Achievements, Top 3 Moods Logged, Top 3 FitPartners, Last 3 Images Posted from a FitRoutine Log, # of FitChallenges Participated In.

The goal is to share insights to the sales/service/marketing user on accomplishments, giving motivation to improve job KPIs in view of fitness achievements.

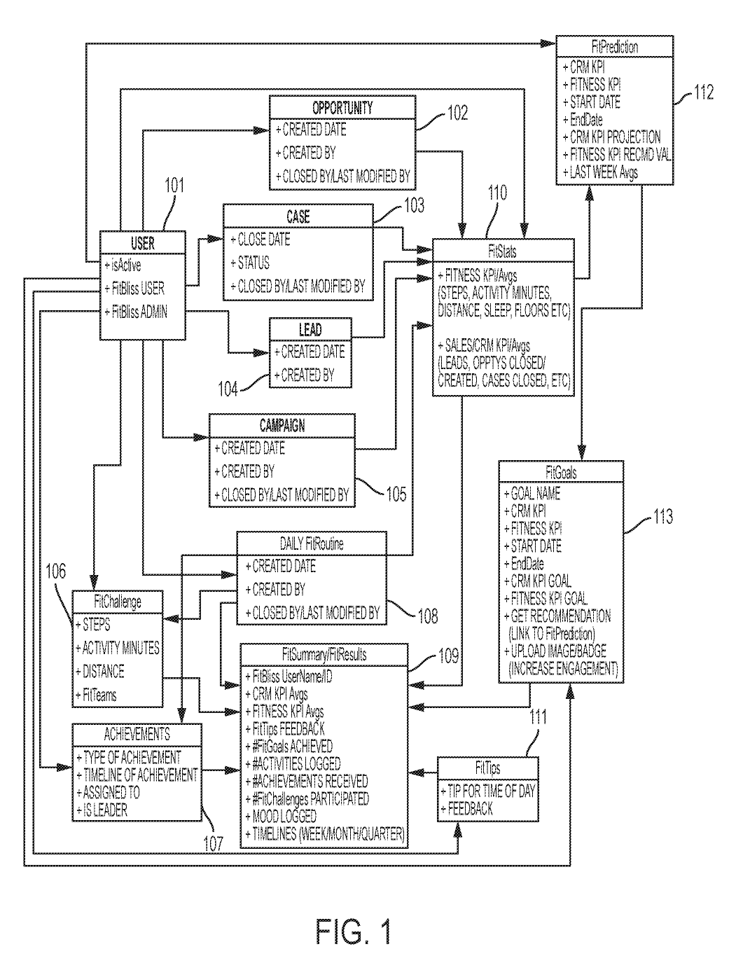

FIG. 1 shows schematic representations of classes/objects used in programming the FitBliss platform, under an embodiment. The FitBliss may provide the following classes/objects (shown in FIG. 1) as further described below.

### User (101)

The User is a person who has access to a Salesforce.com license. Users are considered ‘active’ when users' license is turned on and users to log into their Salesforce account.

FitBliss User is a permission set that FitBliss enables for the users who will get access to the FitBliss product.

FitBliss Admin is a permission set that FitBliss enables for the users who will get both access to the FitBliss product as well as Administrative rights to support the FitBliss product inside Salesforce.com.

### Opportunity (102)

Salesforce ‘Sales Cloud’ technology provides users access to the Opportunity Object which is used to manage the sales cycle in the ‘opportunity’ stage. Salesforce users who have access to the Opportunity object will be able to ‘Create’ an opportunity based on an ‘Account’ record or ‘Lead Conversion’ to ‘Opportunity’.
Created Date is the date in which the Salesforce user, typically a sales rep, may CREATE the opportunity inside Salesforce.com.
Created By is the Salesforce User who created that Opportunity inside the Opportunity Object.
Closed By/Last Modified By provides the details on who closed the Opportunity from current stage to closed. This provides information on who the ‘owner’ of the opportunity is after typically moving from an inside sales rep to the field sales rep.

### Case (103)

Salesforce ‘Service Cloud’ technology provides users access to the ‘Case’ Object which is used to manage the service ticketing cycle in the ‘service ticket’ stage. Salesforce users who have access to the Case object are able to manage the case cycle within Salesforce.com.
Close Date is when the service engagement stage is ‘closed’. This change in stage has a date assigned to it. This date is the ‘Close Date’.
Status is the stage in which the Case is currently. This can be ‘open’ in progress' ‘closed’ or other depending on the customer's instance of Service Cloud.
Closed By/Last Modified By provides the details on who closed the Case from current stage to ‘closed’. This provides information on who the ‘owner’ of the case is.

### Lead (104)

Salesforce ‘Sales Cloud’ technology provides users access to the Lead Object which is used to manage the sales cycle in the ‘lead’ stage. Salesforce users who have access to the Lead object are able to ‘Create’ a lead based on an ‘Account’ record.
Created Date is the date in which the Salesforce user, typically a sales rep, may CREATE the lead inside Salesforce.com.
Created By is the Salesforce User who created that Lead inside the Lead Object.

### Campaign Object (105)

Salesforce ‘Sales Cloud’ technology provides users access to the Campaigns Object which is used to manage the marketing cycle in the account. Salesforce users who have access to the Campaign object are able to ‘Create’ a campaign based on an ‘Account’.
Created Date is the date in which the Salesforce user, typically a marketing rep, may CREATE the campaign inside Salesforce.com.
Created By is the Salesforce User who created that Campaign inside the Campaign Object.
Closed By/Last Modified By provides the details on who closed the Campaign from current stage to ‘closed’. This provides information on who the ‘owner’ of the campaign is.

### FitChallenge (106)

FitChallenge is a FitBliss technology that allows FitBliss users to create a fitness/wellness related challenge that is typically related to specific type of key performance indicator.
Steps is a type of FitChallenge that tracks steps within a team of I or more.
Activity minutes is a type of FitChallenge that tracks activity minutes within a team of 1 or more.
Distance is a type of FitChallenge that tracks the distance gone within a team of 1 or more.
FitTeams are teams of the FitBliss participants.

### Achievements (107)

Achievements is a FitBliss technology that allows FitBliss users to receive an achievement image on the user's profile that is aligned with a metric that was achieved by the user. An example would be to provide the user with ‘Monkey’ achievement when the user takes 50,000 steps in a week. This achievement is recorded against each FitBliss user who attains that metric during the Sunday through Saturday start and end dates.
Type of Achievement is specific to ‘steps’ distance“calories burned” activity minutes' ‘sleep’ floors' and more that are tracked within FitBliss.
Timeline of Achievement is daily, weekly, monthly, lifetime (1-time)
Assigned to is the FitBliss user who achieves that achievement. This is a FitBliss user.
Is Leader is an achievement award given to a leader during the timeframe of an achievement. The example would be a weekly achievement and the leader of ‘steps’ achievement would receive the ‘Leader’ achievement.

### Daily FitRoutine (108)

Daily FitRoutine is a FitBliss technology that captures all the activities done in a specific day. This captures things like steps, basketball, swimming, meditation, distance covered, calories burned, activity minutes, sedentary minutes, and more.
Created Date is the day of the Daily FitRoutine
Created By is the owner of the Daily FitRoutine. This is typically the FitBliss user.
Closed By/Last Modified is the owner of the Daily FitRoutine. This is typically the FitBliss user.

### FitSummary/FitResults (109)

FitSummary/FitResults is a FitBliss technology that brings together all the data into one stream of information giving the user a snapshot of results against FitBliss data and Salesforce data coming from Opportunity, Case, Lead, and Campaign.
FitBliss User name
CRM KPI Averages
Fitness KPI Averages
FitTips Feedback
FitGoals Achieved
Activities Logged
Achievements Received
FitChallenges Participated in
Mood Logged
Timelines (week/month/quarter)

### FitStats (110)

FitStats is a FitBliss technology that creates the averages and analysis of the CRM Data coming from the Salesforce Objects and from the FitBliss technology. This is where the calculations are being generated to understand how to integrate data with FitGoals technology and the FitPrediction Technology. This also helps feed the FitSummary/FitResults object. FitStats may include the following data.
Fitness KPIs

1. Steps, 2. Activity Minutes, 3. Floors, 4. Distance, 5. Sleep, 6. Sedentary Minutes, 7. Calories Burned, 8. Colleagues Tagged, 9. Mood Logged, 10. Location of Activities, 11. Commutes (e.g. via bike versus automobile).
Sales CRM KPIs

1. Leads Created, 2. Opportunities Created, 3. Opportunities Closed, 4. Cases Closed, 5. Campaigns Created, 6. Campaigns Closed.

### FitTips (111)

FitTips is a FitBliss technology that populates health & productivity related recommendations personalized to employee behavior and engagement. This includes FitBliss' machine learning technology that learns the behaviors of the FitBliss user. The machine learning technology may capture every type of engagement with the FitBliss platform and produce FitTips that are highly accurate to the employee's interests, lifestyle, job role, location, colleagues, etc.
FitTips may be provided based on the time of the day
FitTips may have a feedback mechanism that tracks engagements to build personalized recommendations for the user.
FitTips may be provided within the Salesforce technology platform in the selected Objects from the FitBliss Admin as well as the Salesforce users, including Salesforce Admins.
FitTips may populate all types of health & productivity related content that is cross-correlated to optimize the health & productivity of each FitBliss user.

These FitTips are generated by FitBliss in a pool of FitTips that will constantly evolve & improve as the collection of data starts to populate and feedback is generated within the FitBliss application.

### FitPrediction (112)

FitPrediction is a FitBliss Technology that starts to predict the outcomes of fitness activity and CRM-related productivity based on historical data of the user including Leads/Opportunity/Case/Campaigns and FitBliss technology. This generates data that feeds into FitGoals.
CRM KPI
Fitness KPI
Start Date
End Date
CRM KPI Prediction
Fitness KPI Recommended Value
Last Week Averages
Last Month Averages
Last Quarter Averages
Last Year Averages

### FitGoals (113)

FitGoals is a FitBliss technology that is the core front end of the FitProductivity suite. This is where the user can create FitGoal for themselves. This includes a FitGoal name, Difficulty level (easy, medium, hard, ultrabliss, custom), type of KPI both for crm and fitness, start and end date, and values either generated by selecting a Difficulty type or a custom value populated by the user. FitGoals are created by FitBliss users.

FitBliss may track all the FitGoals created and analyze the accuracy levels and provide updated values as the FitGoals start to show levels of accuracy on a selected FitGoal vs the actual outcome of the FitGoal.

Each FitGoal provides achievement awards on the levels of achievement. FitBliss provides Bronze, Silver, Gold, and Platinum
Bronze achievement is achievement of one KPI at 70-84% and the other at 100%
Silver is achievement of one KPI at 100% and the other at 85-99%
Gold is achievement of both KPIs at 100%
Platinum is achievement of both KPIs at 101% and up.
Each Achievement is then populated on the FitResults page for the FitBliss User to Access

Since there are 2 KPI (health/productivity) goals under an embodiment, the FitBliss platform may provide these achievements based on how well the user performs based on % of goal achieved. If a user achieves 1 goal at a percentage over 100% and the other is within 70-84% (meaning one goal was achieved at 100%, and the other wasn't), then the user receives the Bronze Achievement. After receiving this type of Achievement, a user may access the Achievement on the FitResults page to access the history of FitGoals a user has achieved at least Bronze up to Platinum.

FitGoals may have a page that allows the user to create as many FitGoals as he/she would like.

The FitGoals technology may incorporate FitTips that are most relevant to FitGoals.

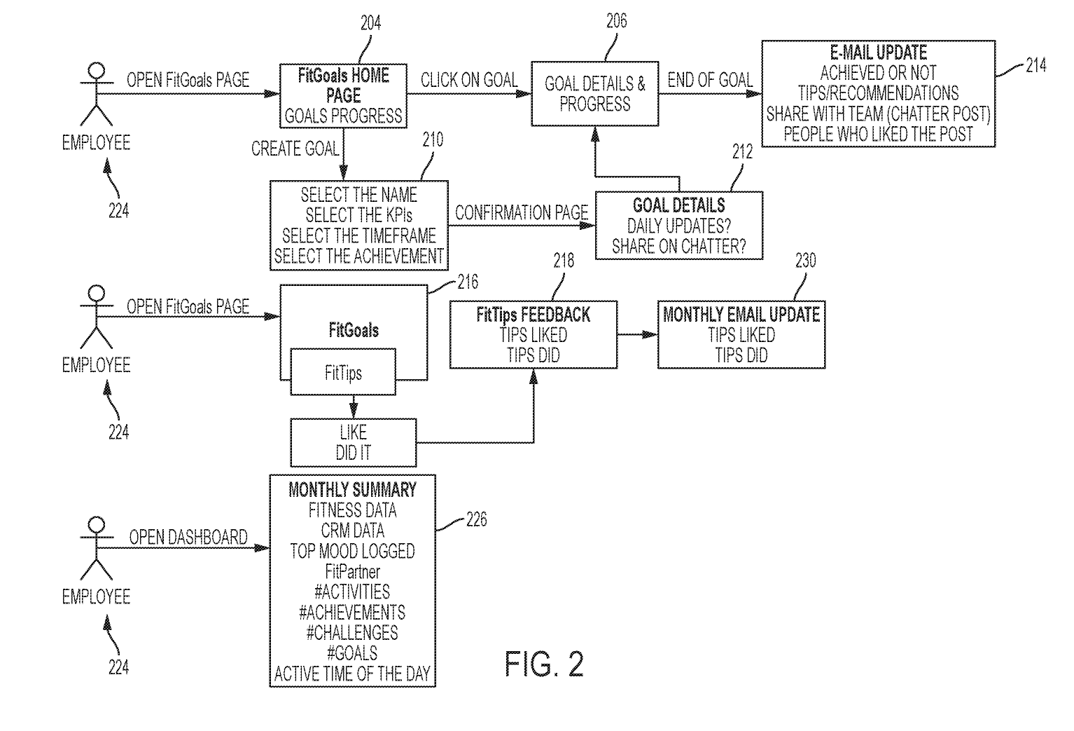

FIG. 2 shows provides an example of FitProductivity workflow. A user 224 may open the FitGoals page to reach the FitGoals Home Page 204 (which may provide general goal progress data). A user 224 may direct the interface to a Goal Details and Progress page 206 (by selecting a particular goal). Alternatively, a user may click through to a create goals page 210 allowing user to select the name of a goal, select KPIs, select time frame, and select achievement. Upon creating a goal, the workflow directs a user to a confirmation page 212 which provides the user with Goal details 212. The page allows the user to elect receipt of daily email updates 214 (e.g. the user may elect the “Track your progress with daily email updates” option) and an option to motivate progress through a social media team concept (e.g., the user may elect the “Get motivated with your team support, post it on chatter” option). In particular a “post it on chatter” link may itself provide clickable access to one or more social media sites including Chatter. Email updates 214 may include fitness goal progress, levels of achievement, FitTips, and an ability to share goal data via social media, under an embodiment.

FIG. 2 also shows that a user 224 may access a FitGoals page 216 which may include a FitTips section. The user may use the FitTips section to “like” tips or report compliance with a tip by clicking “Did It”. A user may click through to a FitTips Feedback page 218 which provides information regarding tips liked or performed. The FitProductivity platform may also forward the user an email 230 summarizing tips liked/performed or providing new tips.

FIG. 2 provides user 224 access to a Monthly Summary page 226. The monthly summary page may provide some or all of the following information: Fitness Data, CRM Data, Top Mood Logged, FitPartner, #Activities, #Achievements, #Challenges, #Goals, Active time of day, etc.

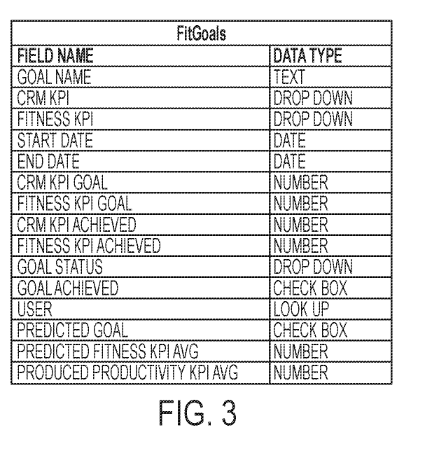

FIGS. 3-7 show data collected in the FitBliss platform, under an embodiment. FIGS. 3-7 may also represent schematic representations of classes/objects used in programming the FitBliss platform, under an embodiment.

FIG. 3 shows data collected with respect to FitGoals. The table shown in FIG. 3 shows a FitGoals data table including Field Name and Data Type.

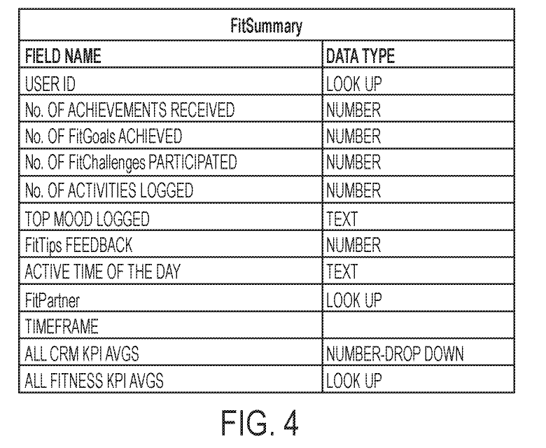

FIG. 4 shows data collected with respect to FitSummary. The table shown in FIG. 4 shows a FitSummary data table including Field Name and Data Type.

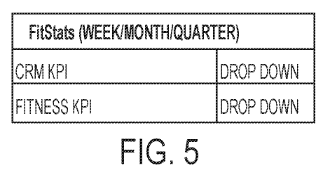

FIG. 5 shows data collected with respect to FitStats. The table shown in FIG. 5 shows a FitStats data table including Field Name and Data Type.

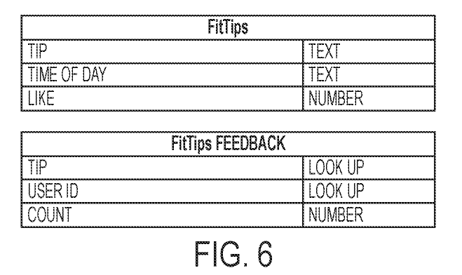

FIG. 6 shows data collected with respect to FitTips and FitTips Feedback. The table shown in FIG. 6 shows a FitTips and FitTips Feedback data table including Field Name and Data Type.

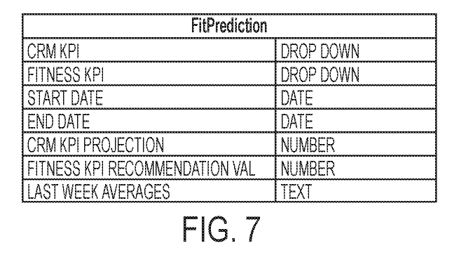

FIG. 7 shows data collected with respect to FitPrediction. The table shown in FIG. 7 shows a FitPrediction data table including Field Name and Data Type.

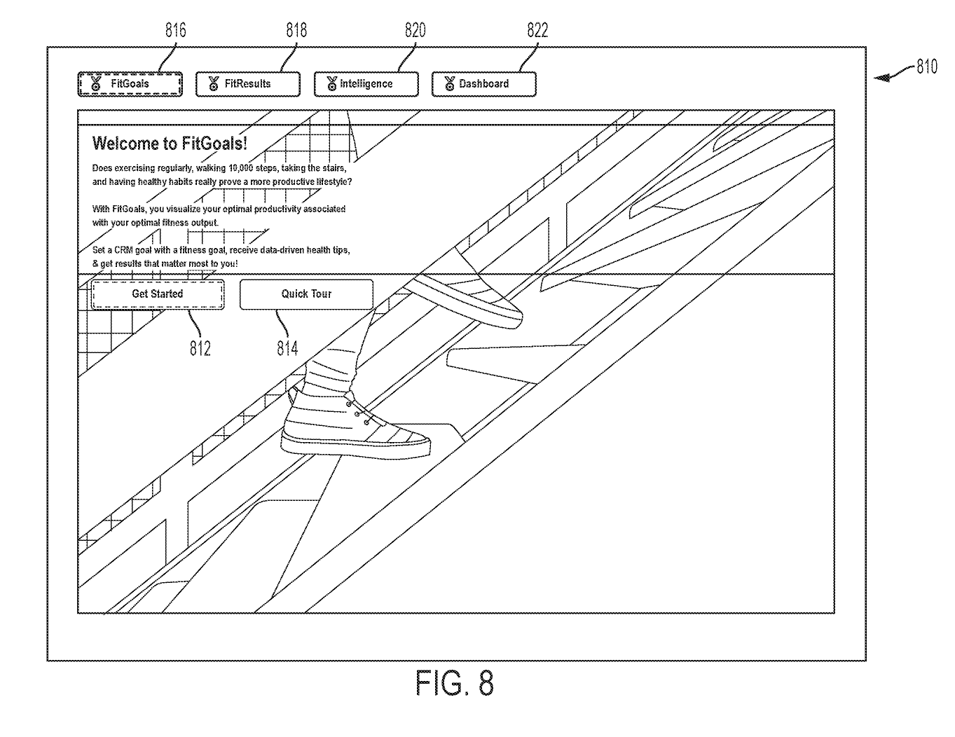

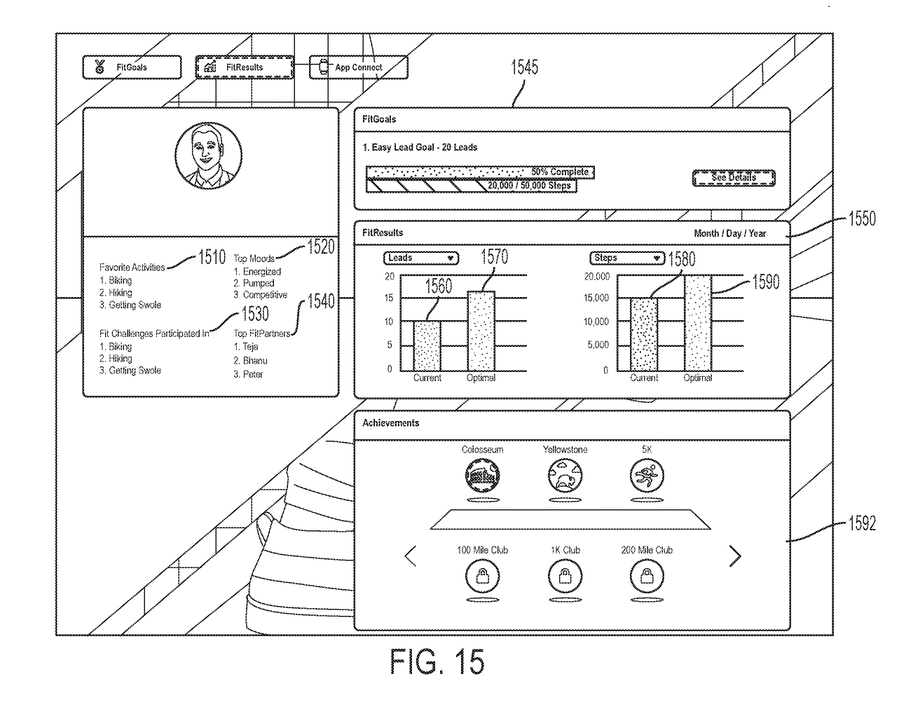

FIG. 8 shows an introduction page 810. The introduction page features a FitGoals indicator 816, a FitResults indicator 818, Intelligence indicator 820, and a Dashboard indicator 822. The indicators inform the user of the user's general location in the FitProductivity workflow. Note that the FitGoals indicator is marked as ‘selected’ throughout the FitGoals workflow (FIGS. 8-14). The FitResults indicator is highlighted when the user reaches a FitResults page (FIG. 15). The Intelligence indicator 820 corresponds to pages providing user analytics and insights based on historical user fitness and company productivity metrics. The Dashboard indicator corresponds to real-time charts on logged activities (both FitConnect and Routine Log details). The user can only click on FitResults after completing at least one FitGoal (meaning creating a FitGoal and waiting until the completion of the FitGoal).

As indicated above, FIG. 8 shows an introduction page 810. A user may take a quick tour 814 of the FitProductivity application or may select Get Started 812 to set a fitness goal.

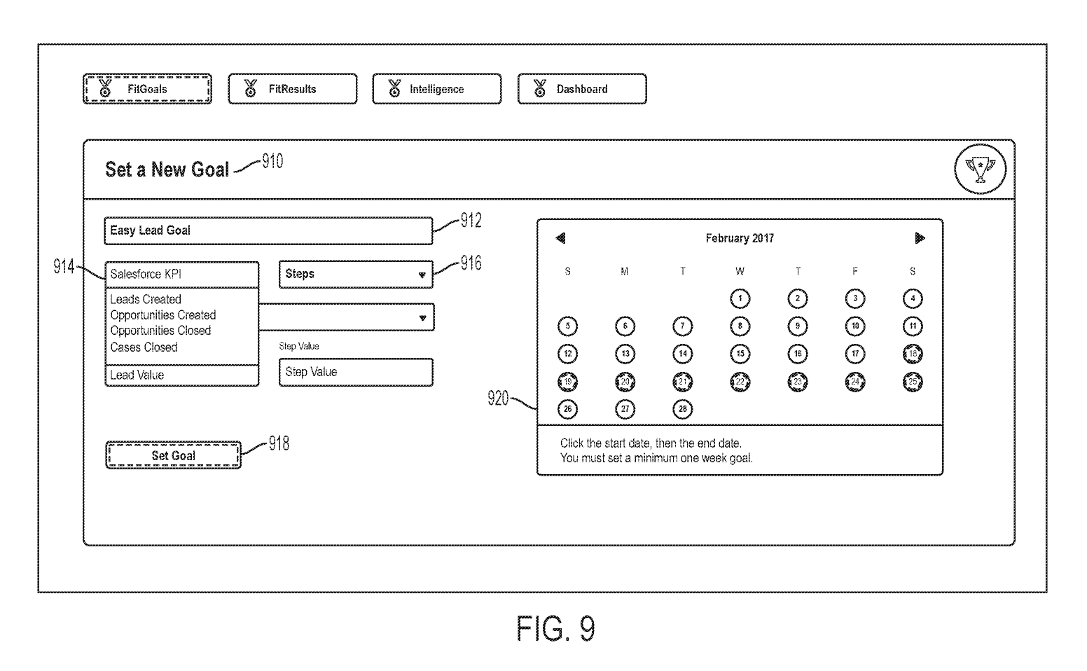

FIG. 9 shows an interface for setting a new goal 910. The upper left of the interface shows a text box 912 which user may use to enter a label. Here the text box features the “Easy Lead Goal” label. The interface features a drop down menu 914 showing lead options including: leads created, opportunities created, cases closed, etc. The interface then allows user to pick Fitness & Wellness Key Performance Indicators (KPIs) using drop down menu 916. The user may select steps, distance, activity minutes, sleep, water intake, floors, and more. Here the user has selected steps. The user must also indicate a time period 920 for completion of the goal. With reference to this example, the user selects February 18-25. The user may then select Set Goal 918 to proceed.

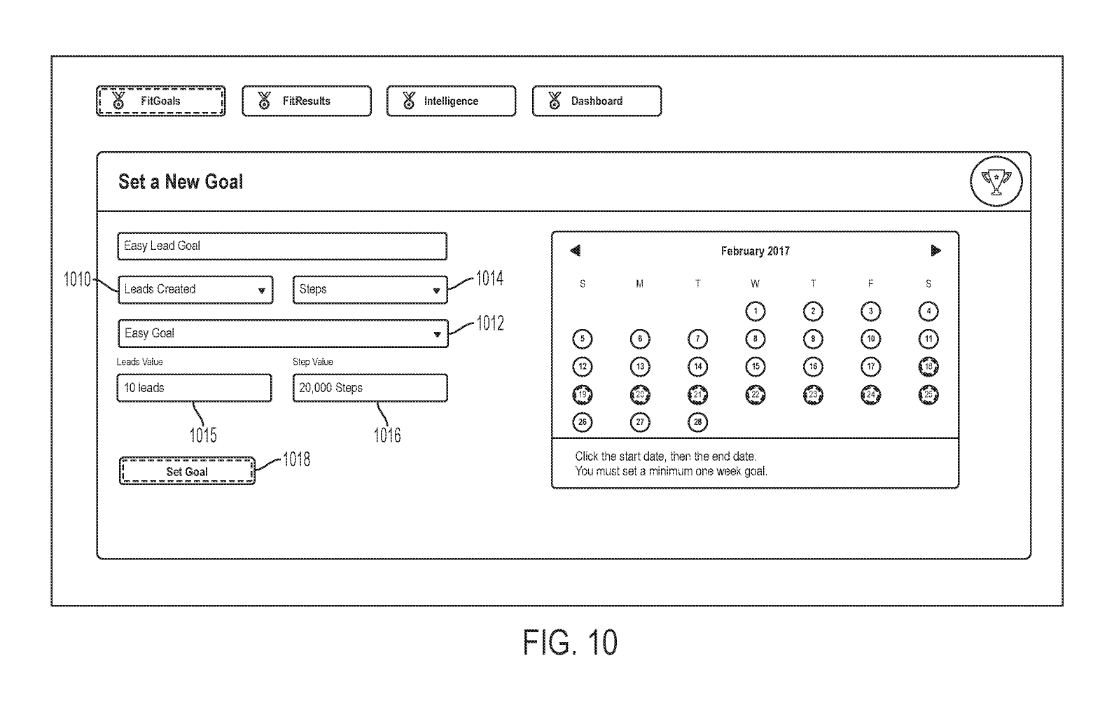

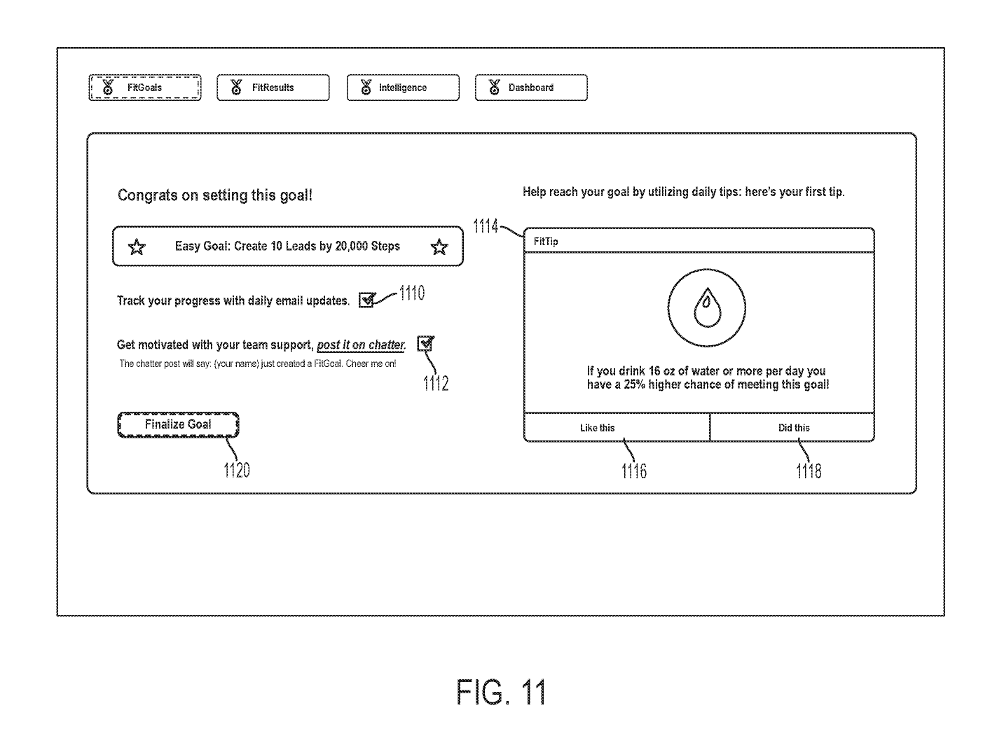

FIG. 10 shows the same page with productivity goal options available through drop down menu 1010. The user may indicate easy goal, medium goal, hard goal, or Ultra Bliss goal using drop down menu 1012. Here the user has selected steps as a fitness goal using menu 1014. The fields 1015 and 1016 automatically populate based on the selected goal. Easy goal populates fields based on 75 percent of user's historical fitness and CRM data, e.g. fitness and CRM data from the prior week. Medium goal populates fields based on 100 percent of user's historical fitness and CRM data, e.g. fitness and CRM data from the prior week. Hard goal populates fields based on 125 percent of user's historical fitness and CRM data, e.g. fitness and CRM data from the prior week. Ultra Bliss populates fields based on 150 percent of user's historical fitness and CRM data, e.g. fitness and CRM data from the prior week. Historical fitness and CRM data may comprise optimal performance data under an embodiment. These percentages are flexible and may be altered based on selected fitness type. Also, one embodiment allows the user to enter goals directly. The user then sets the goal 1018 and reaches the congratulations page of FIG. 11.

The page shown in FIG. 11 allows the user to elect receipt of daily email updates 1110 (e.g. the user may elect the “Track your progress with daily email updates” option) and an option 1112 to motivate progress through a social media team concept (e.g., the user may elect the “Get motivated with your team support, post it on chatter” option). In particular, a “post it on chatter” may itself provide clickable access to one or more social media sites including Chatter. The page indicates that the “The chatter post will say: (your name) just created a FitGoal, Cheer me on!”. The page also features a FitTip 1114: “If you drink 16 oz of water or more a day you have a 25% higher chance of meeting this goal!” The user may like 1116 the tip, or report accomplishing/following 1118 the tip. The user may then finalize the goal 1120.

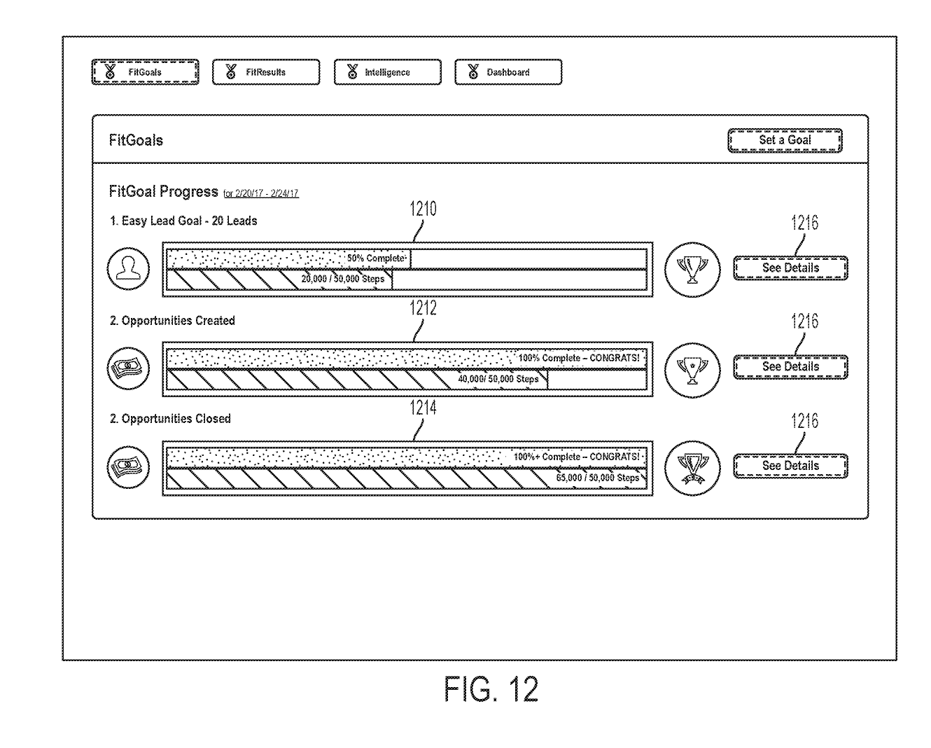

FIG. 12 shows a FitGoals summary page showing goals and completion percentage. The page also shows corresponding completion of fitness goals. The page shows 1210 an Easy Lead Goal of 20 Leads at 50% completion (and at 20,000/50,000 steps). The page shows 1212 an Opportunities created goal at 100 percent completion (and at 40,000/50,000 steps). The page shows 1214 an Opportunities closed goal at 100 percent completion (and at 65,000/50,000 steps). A user may select a Details button 1216 to see additional details regarding each individual goal.

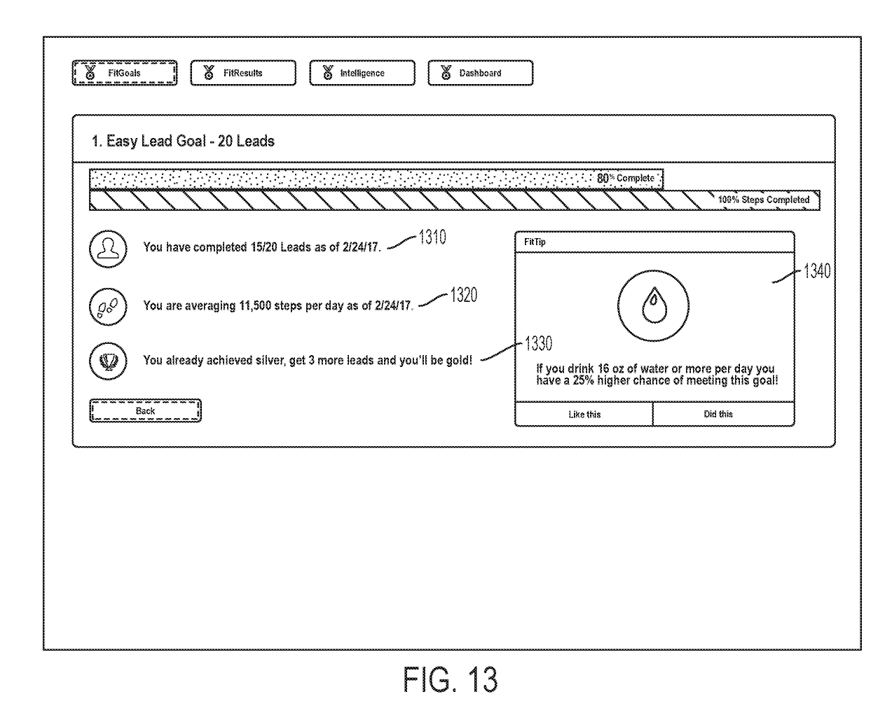

FIG. 13 shows an email sent to user summarizing progress with respect to a goal. Here the screen shows 1310 that the user has completed 15/20 leads as of 2/24/17. The screen also shows that the user 1320 is averaging 11,500 steps per day as of 2/24/17. The screen 1330 encourages the user to get just 3 more leads to move from silver to gold status. The screen also shows a FitTip 1340.

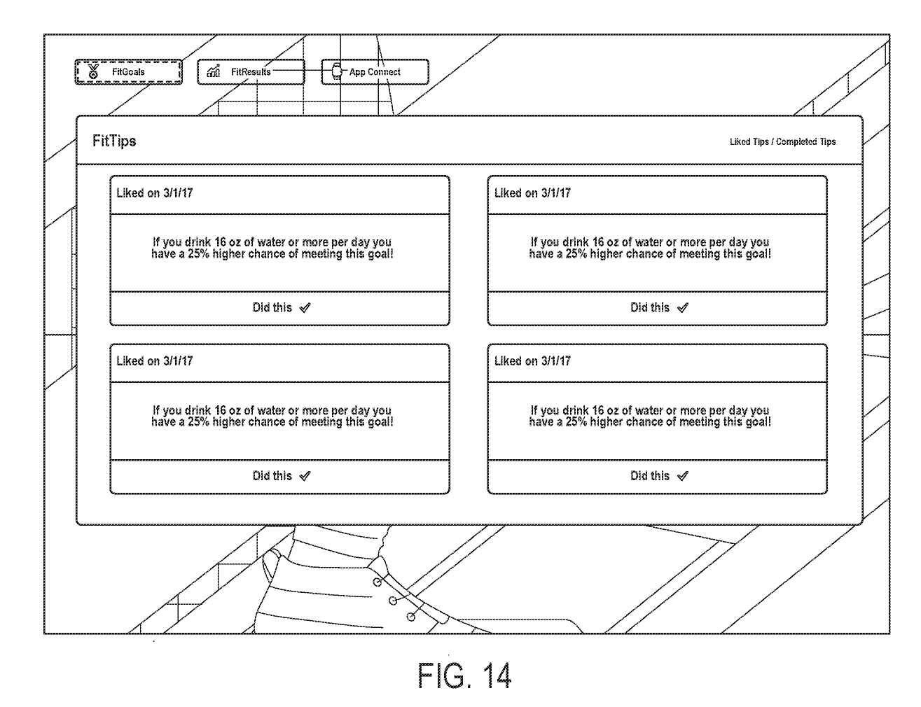

FIG. 14 shows a FitTips page that summarizes tips either liked by a user or marked as completed by a user. Here the page shows four duplicative tips but embodiments may of course track various tips liked/completed in real time.

FIG. 15 shows a summary page for a user. The page shows the user's favorite fitness activities 1510 (e.g. biking), top moods 1520 (e.g. energized, pumped!!), FitChallenges 1530 (e.g. hiking), and Top FitPartners 1540 (e.g. Teja). The page shows fitness progress 1545 in a manner analogous to FIG. 12. The page also shows FitResults 1550. The FitResults component shows productivity and fitness achievements versus optimal targets. The information includes current 1560 versus 1570 optimal leads and current 1580 versus optimal 1590 steps. The summary page also includes an achievement component 1592 showing accomplishment of certain fitness achievements.

FitTips may be time-based, user-feedback oriented, and are intelligently provided based on the user's historical behaviors including and not limited to: sleep patterns, workout patterns, food and dietary choices, location-based data, time of workouts, workout partners, gym memberships, workout class schedule, mood and results of workout, calories burned, and activity correlated with selected productivity levels.

The platform may then mine the data stored and continuously provide more intelligent recommendations that contribute to better outcomes. Outcomes are based on the selected health/wellness/fitness key performance indicators (KPIs) as well as the productivity KPIs

FitProductivity extends to all employees at an enterprise across all measurable employee KPIs. For example a CPA may need to complete tax submissions by a certain date, or an attorney who needs to log a certain amount of billable hours. Both such persons may need to see those KPIs against their health and wellness KPIs that FitBliss provides.

Under an embodiment, the FitBliss platform may comprise FitAssistant. The FitBliss platform may provide FitAssistant as a personalized health management suite based on health behaviors data tracking by wearable devices and health tracking apps. In a modern environment, long sedentary time, eating unhealthy food, high amounts of unhealthy stress and sleep deprivation among employees in the workplace has exacerbated weight management problems. Weight management continues to be a high concern for employers and employees, impairing productivity and increasing health insurance burdens as a direct result.

By splitting ambitious health goals (start with weight) into smaller achievable weekly plans, studying exercise patterns, predicting future health (steps) trends and recommending personalized health content (digital), FitAssistant provides real-time services powered by scalable machine learning and Artificial Intelligence (AI) technology. The potential target customers include not only employees in the workplace, but also patients after knee replacement, hip replacement and other surgeries in hospital.

From a technical view, the FitBliss platform (including the FitAssistant platform) provides a million-user-level scalable health pattern prediction technology, benefiting people (patients and employees) all over the world.

### Weight Management Solution

### 1. Exercise Pattern Study

### 1.1 Data Source

The FitBliss platform imports health behavior data under one embodiment from users' wearable devices or health tracking apps, such as Apple Watch™, Fitbit™, Strava™, Jawbone™, Runkeeper™, Misfit™, Mapmyfitness™, MyFitnesspal™ etc. Users under one embodiment synchronize their health data through AppConnect™ (integration portal), and the data from different resources are integrated together through Human API (integration partner) or directly into FitBliss via the Wearable/App API, allowing us to access digital structured health data from devices and apps in real time.

The information used in an exercise pattern study includes “User”, “Date” and “Steps”, under an embodiment. User “Id” works as a distinct external identifier for each record. No two records share the same “Id” in the database of Salesforce.

### 1.2 Data Preprocessing—Generating Clean and Accurate Data (Due to Multiple Wearables and Apps by One User)

The first step before studying exercise patterns is under one embodiment to remove missing values. Missing values are caused for example by the absence of data, partly because users did not synchronize their data or because user's inconsistent use of wearable trackers. Leaving missing values without processing them may skew the analysis result.

One strategy under an embodiment imputes missing values with the mean of historical number of steps if non zero. For example, the table below reflects the steps data of a user from January 1st to January 9th. There is no data showing up in January 3rd and January 6th.

| | Date | | | | | | | | |
|---|---|---|---|---|---|---|---|---|---|
| | 01/01 | 01/02 | 01/03 | 01/04 | 01/05 | 01/06 | 01/07 | 01/08 | 01/09 |
| Steps | 10,324 | 12,398 | NaN | 9,415 | 9,823 | NaN | 16,231 | 8,917 | 6,342 |

After calculation, we get the mean value of steps data from January 1st to January 9th, 10493, which will be the steps data we impute for that in January 3rd and January 6th. The table below shows what the data looks like after imputation.

| | Date | | | | | | | | |
|---|---|---|---|---|---|---|---|---|---|
| | 01/01 | 01/02 | 01/03 | 01/04 | 01/05 | 01/06 | 01/07 | 01/08 | 01/09 |
| Steps | 10,324 | 12,398 | 10,493 | 9,415 | 9,823 | 10,493 | 16,231 | 8,917 | 6,342 |

Moreover in order to lessen the heavy load of processing, the FitBliss platform uses under an embodiment the last 100 days data of each user as the predictors for future steps.

### 1.3 Exercise Pattern Study

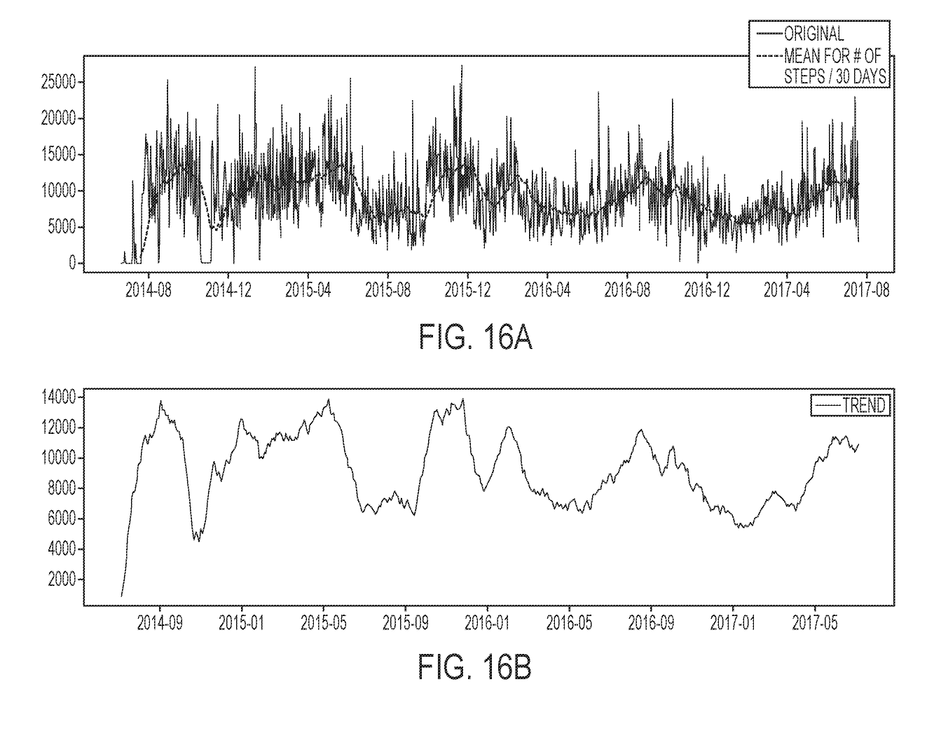

There may be big fluctuations for “the number of steps” people take every day, which causes difficulty in figuring out how “the number of steps” trends. FIG. 16A shows steps (y-axis) per day (x-axis). The solid and dotted lines show unfiltered and filtered data, respectively. FIG. 16B again shows steps (y-axis) per day (x-axis). However each data point comprises a thirty (30) day moving average. In this way FIG. 16B shows how steps trend over time.

The method used to decompose the steps data into trend, seasonality and residual comprises under one embodiment the additive model: Y[t]=Trend[t]+Seasonality[t]+Residual[t]. “Trend” is the varying mean over time. A moving average is commonly used to smooth out short-teal fluctuations and highlight longer-term trends or cycles.

“Seasonality” and “Residual” are described as follows. “Seasonality” is the variations at a specific time frame. The seasonal component is first removed by applying a convolution filter to the data. “Residual” is arrived by Y[t]−Trend[t]−Seasonality[t].

By the method described above, users may figure out how exercise patters manifest over time.

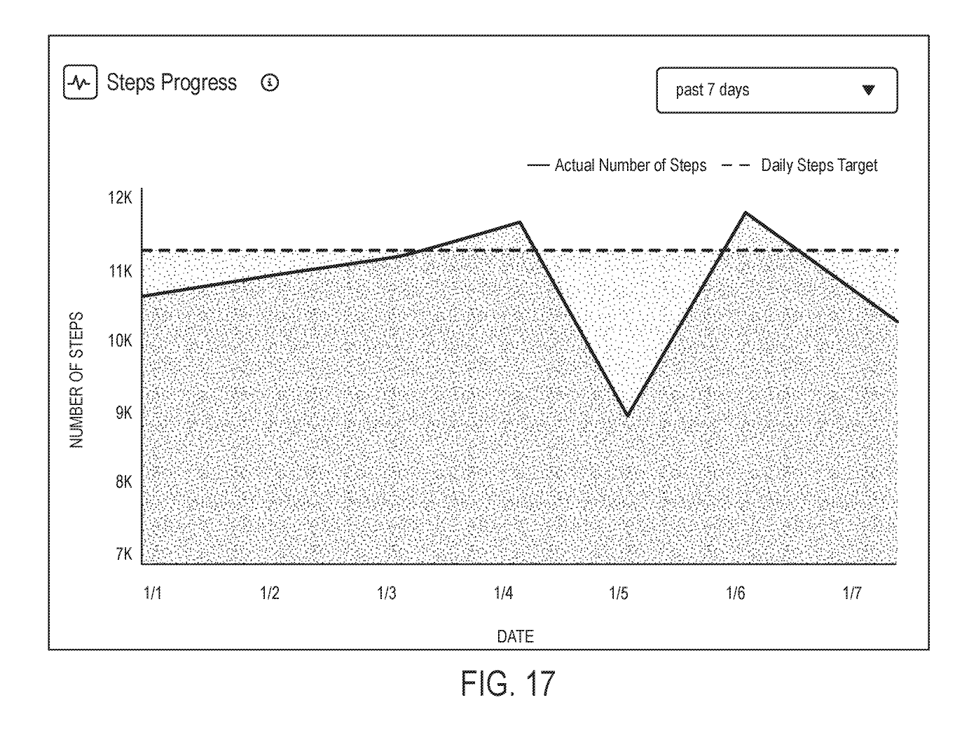

FIG. 17 shows a frontend display illustrating exercise steps trend and daily steps target. Users have flexibility to choose the length of time period ranging from last 7 days, 30 days, 60 days, and 90 days.

### 2. Steps Recommendations—Health Recommendations

Since wearable devices provide a convenient way to quantify intensity of users' daily activity, FitAssistant focuses on the steps users take as the starting point of a weight management solution.

Assume under one embodiment that users have three categories of weight management goals: lose weight, maintain weight, and gain weight. These goals depend on the relationship between calories intake and calories consumption every day. In order to help users lose weight or maintain weight successfully, the FitBliss platform provides under an embodiment solutions on how to spend extra amount of calories through taking steps.

A user may ask the following question “What's the extra amount of calories that I need to expend and how many steps do I need to take?”.

There is in general a minimum calories needed to maintain body weight. Here is where the concept of BMR (Basal Metabolic Rate) applies. Basal Metabolic Rate is the estimated number of calories a person may consume in a day to maintain their body-weight assuming they remain at rest.

For men, BMR=4.5*weight (lbs)+190.5*height (ft)−5*age (y)+5

BMR=10*weight (kg)+6.25*height (cm)−5*age (y)+5

For women, BMR=4.5*weight (lbs)+190.5*height (ft)−5*age (y)−161

BMR=10*weight (kg)+6.25*height (cm)−5*age (y)−161

As one example, for a male with 25 year-old age, 5 feet 11 inch height and 200 lbs weight, he needs at least 1908 calories per day to maintain his weight. In addition, absolute resting state is an ideal state. Therefore the model usually applies a multiple to the basic calories requirement. Under an embodiment a multiple of 1.2 is applied resulting in 2289 calories considering a real world environment. If he intakes 2500 calories from food per day, he needs to spend 211 calories per day by exercise (walking and running) to maintain his weight. 211 calories is obtained by the subtraction between calories intake and basic calories need (211 calories to spend=2500 calories intake −2289 calories needed).

It should be noted that 1 pound of body weight, approximately 0.45 kg, equals about to 3500 calories, making it easy to transform between calories to be spent and weight to be lost based on the fact. As an example, assume a user wants to lose 1 lb in weight, in other words, 0.45 kg and 3500 calories. Spending such amount of calories within 1 or 2 days is not generally reasonable. FitAssistant solves the problem by helping split the goal into smaller intervals based on a weekly plan. Spending an extra 3500 calories per week means spending 500 extra calories per day. Therefore how many steps do we need to take in order to realize the calories goal?

In order to transform the calories goal into steps goal, the calories consumption per step is key to the solution. Calories spent per mile depends on the walking speed. Thus as long as we know the calories spent per mile and the number of steps people take per mile, it's easy to deduce the calories burned per step.

Under one embodiment, if a user walks casually, at an assumed speed if 2 miles/hour, the calories burned per mile is 0.57*weight (lbs). Similarly, if a user walks with brisk speed, an embodiment of the systems and methods herein may assume 3.5 miles per hour. The calories burned per mile is then 0.5*weight (lbs). In this way, the number of steps needed to be taken in order to realize the calories goal is arrived through the relationship between extra calories to be burned and the calories spent per step.

This process is illustrated using the following formulas.

1—Casual walking—2 miles/hour

1.1—Calories burned/mile=0.57*weight (lbs)

1.2—The number of steps/mile=total number of steps/total miles

1.3—Calories burned/steps=(calories burned/mile)/(the number of steps/mile)

1.4—The number of steps=calories to be spent per day/(calories burned per step)

2—Brisk or power walking—3.5 miles/hour

2.1—Calories burned/mile=0.5*weight (lbs)

2.2—The number of steps/mile=total number of steps/total miles

2.3—Calories burned/steps=(calories burned/mile)/(the number of steps/mile)

2.4—The number of steps=calories to be spent per day/(calories burned per step)

After conducting outdoor experiments to gauge steps/mile rates, an embodiment of the systems and methods provided herein estimates an average number of steps per mile for two walking speeds. For people who walk casually, an estimate comprises 2222 steps per mile. For people who walk briskly, an estimate comprises 2000 steps per mile.

Assume a user wants to lose 1 lb per week. Let's look at the example taken above. The user needs to spend 211 calories to maintain his weight and 500 calories per day to lose 1 lb per week, in other words, there are 711 calories need to consumed by physical activities every day.

Assume that a user is accustomed to walk casually, the calories burned per mile will be 0.57*200, that is, 114 calories. The calories consumed per step will be 114/2222, that is, 0.0513. The number of steps to take in order to spend 711 calories will be 13859 every day.

The process to calculate steps recommendations for weight maintaining is similar to the description above. As for the users who set weight gain as their goal, FitAssistant transforms the weight gain target as extra calories intake per day and recommends a target daily calories intake for users. Assume there is a user who needs 2000 calories per day for basic metabolism and wants to gain 1 lb of weight per week, that is an extra 3500 calories per week and 500 calories per day. Thus we recommend the user to intake 2500 calories per day to realize the weight goal.

Under an embodiment, a customers may want to achieve large weight loss goals, such as 10 lbs, 20 lbs or more. FitAssistant helps users by splitting the weight goal into smaller achievable ones considering the safe range of weekly weight loss recommended by CDC (Centers for Disease Control and Prevention), by generating reasonable time frames, weekly weight loss goals, daily steps recommendations and calories intake targets.

For the example a current weight of a user may be 148.5 pounds and a corresponding ideal weight is 140 pounds. Based on personal demographic information provided by the user, FitAssistant may generate a weekly plan lasting for 3 months ranging from Dec. 2, 2017 to Mar. 5, 2018, recommending a loss of 0.5 pounds per week with a daily intake of 1900 daily calories and a daily target of 11,300 daily steps.

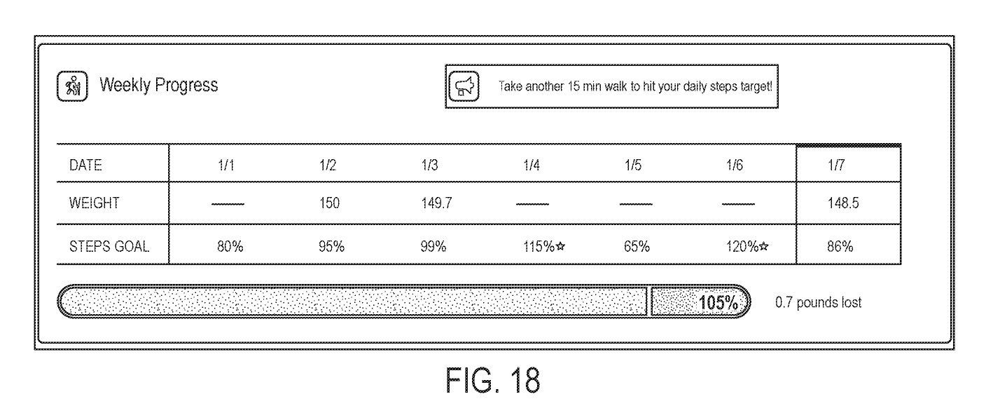

Moreover, the FitBliss platform provides consistent weight progress tracking on a week to week basis, reflecting the positions users are in along the way to their big weight goal. FIG. 18 shows date, weight and steps goal by day of week (1/1-1/7). FIG. 18 shows the updated weight of a user whose data is synchronized with the FitBliss platform through wearable devices or whose data is entered through a FitBliss platform dashboard. FIG. 18 shows the percentage of real number of steps to target steps rendered everyday (1/1-1/7). FIG. 18 also shows a progress bar illustrating the weekly weight goal achievement. FIG. 18 shows that the user has achieved 105% of the target weight loss goal. Note that weight values are not generally populated daily as users typically weigh in once every few days or every other day.

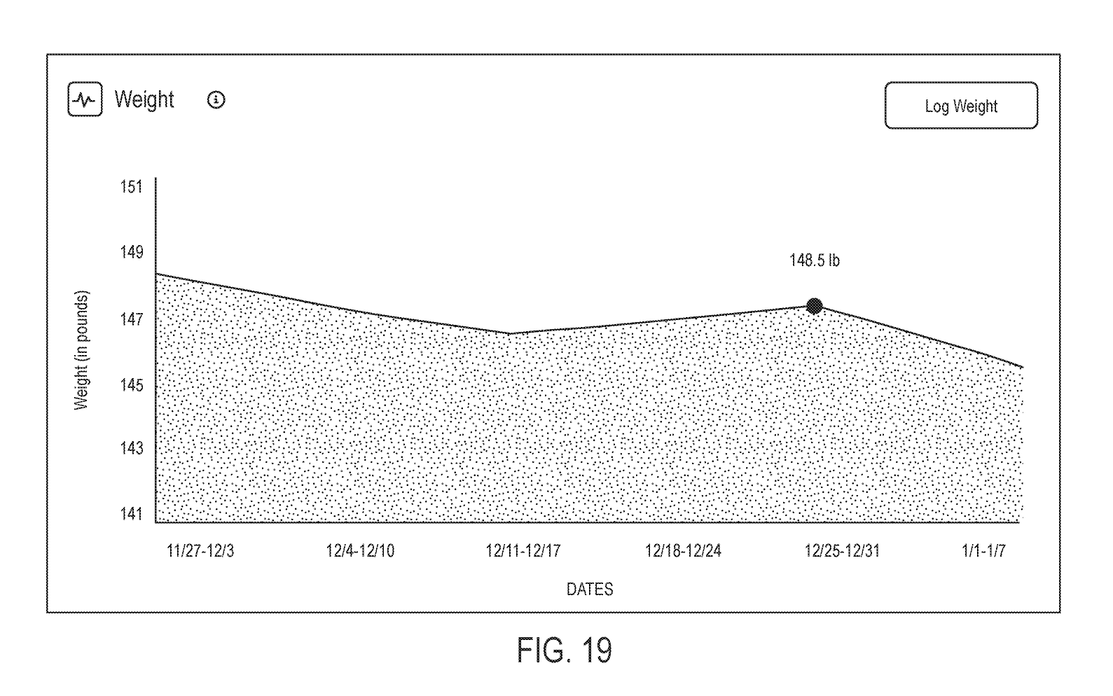

FIG. 19 shows weight gain/loss over time and provides a visual of weekly weight change. Note the FIG. 19 shows weight data on week to week basis. As seen, the user posts a weight of 148.5 pounds for the week of 12/25-12/31. Note that the graph after the 12/25-12/31 week (corresponding to 148.5 pounds) represents a projected weight ‘trend’.

Under one embodiment of FitAssistant, the platform enriches angles of recommendations based on additional metrics including sleep quality, sedentary minutes, calories burned, types of activities, activity minutes, flights of stairs, job title, location and family (single/married) and etc. These multiple factors work together as a system to influence the change of weight.

Sleep is an important modulator of neuroendocrine function and glucose metabolism and sleep loss has been shown to result in metabolic and endocrine alterations, including increased hunger and appetite, as a consequence, thereby increasing the risk of obesity and supporting the use of sleep quality as a recommendation metric.

Of course physical activity (PA) and exercise training (ET) influence weight gain, initial weight loss, and weight maintenance. The FitBliss platform may recommend high volume aerobic exercise training with and without caloric restrictions, under an embodiment.

Job title is also an indispensable factor differentiating the physical activity intensity of different population groups. Employees like engineers, computer programmers and scientific researchers often spend significant time in front of laptops. Longer periods of sedentary time may be more strongly associated with the amount of fat deposited around internal organs, potentially leading to type II diabetes and heart disease. The FitBliss platform may design strategies to facilitate increased physical activity, i.e. using step management goals to manage chronic disease conditions.

Relationship status may be associated with lower body weight. Cohabiters and married respondents tend to weigh more. Even marital status transition may play an important role in weight change. The FitBliss platform uses under one embodiment the relationship between marital status and weight management to recommend right fit tips.

Using health tracking data (steps, sleep quality, flights of stairs and activity minutes) and other factors mentioned above (job title, family and location), the FitBliss platform implements machine learning to optimize weight management. By allowing machines to study and quantify the lifestyle characters of these users, an optimized solution of weight management may be computed, tailoring the personalized lifestyle recommendations for each individual. The FitBliss platform may also consider the walkability of cities in users' resident cities in helping them to realize their goal by recommending the right places to exercise considering texture of ground, traffic, safety and price of gym program.

### 3. Prediction Technology

### 3.1 Introduction to Cross Validation.

In order to assess the performance of the model, cross validation is used for model training, reflecting the general error on an independent dataset. Touching the test set before training may cause the skewness of prediction results. It's also the reason why we usually split an original dataset into training set and testing set respectively. Under one embodiment, the testing set is the data of the last 7 days. The training set is the remaining data.

For more information about cross validation, a technique called “k-fold cross validation” is described below. In k-fold cross-validation, the original sample is randomly partitioned into k equal sized subsamples. Of the k subsamples, a single subsample is retained as the testing data for estimating the accuracy of the model, and the remaining k−1 subsamples are used as training data. The cross-validation is then repeated k times (folds), with each of the k subsamples used exactly once as the validation data. The k results from the folds can then be averaged to produce a single estimation.

### 3.2 Preconditions of the Model

The model of an embodiment used predict future steps trend is ARIMA, Autoregressive Integrated Moving Average Model. However the preconditions to use the ARIMA model is to guarantee the stationarity of datasets before taking further steps. Note that a stationary process has the property that the mean, variance and autocorrelation structure of data do not change over time. Here we use Dickey-Fuller method to test the stationarity of our data.

If the test statistic is less than the critical value 1%, 5% and 10%, the time series can be regarded as stationary in a 99%, 95% and 90% confidence interval respectively. We need to at least guarantee the property of stationary in one confidence interval.

Below is an example of a test result.

Test Statistic: −7.05<Critical Value (1%): −3.50<Critical Value (5%): −2.89<Critical Value (10%): −2.58

Obviously the time series is stationary in all 99%, 95% and 90% confidence interval.

| | |
|---|---|
| Test Statistic | −7.050754e+00 |
| p-value | 5.534664e−10 |
| #Lags Used | 5.000000e+00 |
| No. Observations Used | 9.100000e+01 |
| Critical Value (1%) | −3.504343e+00 |
| Critical Value (5%) | −2.893866e+00 |
| Critical Value (10%) | −2.584015e+00 |

In addition, in order to reflect the exercise habit objectively, the FitBliss platform regards under one embodiment “the moving average of the number of steps per 30 days” as the time series to be predicted in FitAssistant.

A sudden dropping from the usual 10,000 steps per day to 200 steps for 1 or 2 days may not cause immediate harm to health and not reflect the real change of exercise habit. But if the dropping sustains for 30 or more days such that “the moving average of the number of steps per 30 days” indicates an obvious decrease, we consider it as a potential habit change or health issue. This is also the reason the FitBliss platform takes the moving average as the time series to be predicted.

### 3.3 Parameter Obtainment

Not all time series data performs stationary before feeding into training model. In order to make every time series stationary, we took a log transformation before further processing. For the time series showing “un-stationary” after the transformation, we took differencing, even second order differencing until the data gets stationary. More importantly, the order of differencing determines the parameter “d” in the ARIMA model.

For the time series without differencing before putting into model, d=0.

For the time series with one time differencing before putting into model, d=1.

For the time series with second order differencing before putting into model, d=2.

The FitBliss platform commonly sets the parameter “d” in ARIMA model as 0, 1 or 2.

Here is an example for the time series after first order differencing and second order differencing.

After taking first-order differencing, the time series is not stationary. The Test Statistics (−2.293121)>Critical Value (10%): −2.584015>Critical Value (5%): −2.893866>Critical Value (1%): −3.504343.

| | |
|---|---|
| Test Statistic | −2.293121 |
| p-value | 0.174196 |
| #Lags Used | 6.000000 |
| No. Observations Used | 91.000000 |
| Critical Value (1%) | −3.504343 |
| Critical Value (5%) | −2.893866 |
| Critical Value (10%) | −2.584015 |

After taking second-order differencing, the time series become stationary, showing the effectiveness of the method. The Test Statistics (−7.050754e+00)<Critical Value (1%): −3.504343e+00<Critical Value (5%): −2.893866e+00<Critical Value (10%): −2.584015e+00.

| | |
|---|---|
| Test Statistic | −7.050754e+00 |
| p-value | 5.534664e−10 |
| #Lags Used | 5.000000e+00 |
| No. Observations Used | 9.100000e+01 |
| Critical Value (1%) | −3.504343e+00 |
| Critical Value (5%) | −2.893866e+00 |
| Critical Value (10%) | −2.584015e+00 |

Here is a brief introduction to ARIMA model. ARIMA stands for Auto-Regressive Integrated Moving Averages. The predictors depend on the parameters (p, d, q) of the ARIMA model.

The Number of AR (Auto-Regressive) terms (p): AR terms are lags of dependent variable. For instance, if p is 5, the predictors for x(t) will be x(t−1), x(t−2), x(t−3), x(t−4), x(t−5).

The Number of MA (Moving Average) terms (q): MA terms are lagged forecast errors in prediction equation. For instance, if q is 5, the predictors for x(t) will be e(t−1), e(t−2), e(t−3), e(t−4), e(t−5) where e(i) is the difference between the moving average at the instant and actual value.

The Number of Differences (d): the order of differencing to make dataset stationary.

For the parameter p and q in ARIMA model, we use autocorrelation function (ACF) and partial autocorrelation function (PACF) to determine them respectively.

Autocorrelation Function (ACF): It is a measure of the correlation between the Time Series with a lagged version of itself.

Partial Autocorrelation Function (PACF): It is a measure of the correlation between the Time Series with a lagged version of itself but after eliminating the variations already explained by the intervening comparisons. For example at lag 5, it will check the correlation but remove the effects already explained by lags 1 to 4.

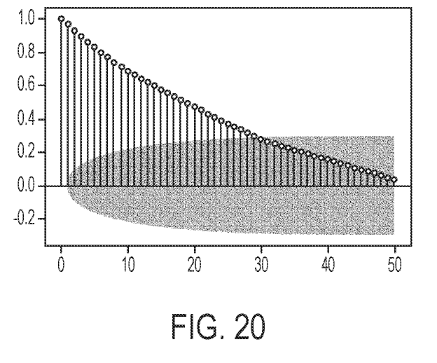

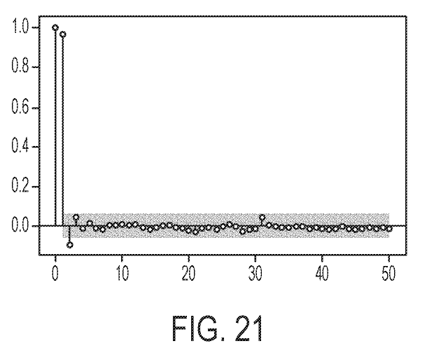

FIG. 20 shows the Autocorrelation Function Plot and FIG. 21 shows the Partial Autocorrelation Function Plot. In these plots, the blue shadow on either side of 0 are the confidence intervals. These can be used to determine the ‘p’ and ‘q’ values.

p: the lag value where the Autocorrelation Function (ACF) chart crosses the upper confidence interval for the first time. In this case, p=29.
q: the lag value where the Partial Autocorrelation Function (PACF) chart crosses the upper confidence interval for the first time. In this case, q=1.

### 3.4 Model Training

After obtaining the required parameters p, d, q in ARIMA model, the method trains the model to perform rolling forecast on “steps trend” users would perform in the next 7 days.

An embodiment then feeds the training set obtained in the first step and parameters obtained into the ARIMA model to train the model. The method appends the predicted data obtained in last step to the training set to predict steps for the next seven days. The process is called a Rolling Forecast.

A rolling forecast is a recurrent process in prediction. Therefore, the last step means the last round of prediction. A rolling forecast is a process that appends predicted results obtained from the last prediction round to the training set to predict results for that of next round.

In order to assess the performance of an ARIMA model, an embodiment uses residuals-fitted plot and density plot of residual errors as important indicators. The residuals here is the vertical distance between the predicted values and real values. The smaller the residuals, the better the model.

If the value of residuals scatters along the fitted values randomly, it shows the residuals of the model roughly have a similar amount of deviation from the predicted values. If the assumption is violated, it indicates there are some patterns/relationships between responsive variables and predictors that are not explained by the model.

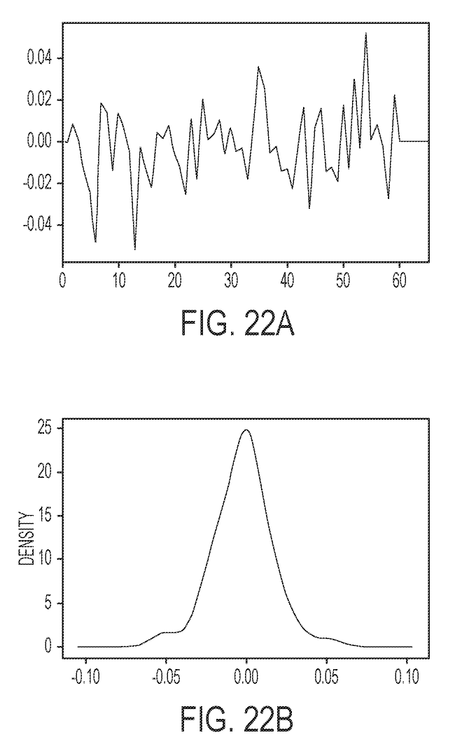

FIG. 22A is an example of a residual-fitted plot. The residuals are y-axis values and the fitted values are in x-axis. The residuals scatter along the fitted values randomly which shows the model explains the variances of the variables well.

Another way to assess the performance of the model is residuals density plot. FIG. 22B shows a residuals density plot with the y axis representing the density of residual values and the x axis representing mean. If it shows a Gaussian distribution with 0 as mean value, we may conclude that there is no bias in the prediction.

Finally we get the predicted values and use Normalized Mean Squared Errors (NMSE) to assess the difference between expected values (testing set) and predicted values.

An embodiment of FitAssistant may extend the steps trend prediction to longer periods of time including 14 days, 30 days, 60 days, 90 days, etc., keeping consistent service along the whole health management cycle. Data shows that the long short-term memory (LS™) model through recurrent neural network performs robust in time series analysis, learning the most important past behaviors and understanding whether or not those past behaviors are important features in making future predictions.

The long short-term memory network may be trained using backpropagation through time thereby overcoming the vanishing gradient problem. As such, it in turn can be used to address difficult sequence problems in machine learning and achieve state-of-the-art results.

### 3.5 Results

Below is an example of the expected values and predicted values after rolling forecast.

Predicted Number of Steps

| | |
|---|---|
| 2017 Aug. 4 | 7748 |
| 2017 Aug. 5 | 7427 |
| 2017 Aug. 6 | 7428 |
| 2017 Aug. 7 | 7200 |
| 2017 Aug. 8 | 7160 |
| 2017 Aug. 9 | 6987 |
| 2017 Aug. 10 | 6972 |

Real Number of Steps

| | |
|---|---|
| 2017 Aug. 4 | 7527 |
| 2017 Aug. 5 | 7397 |
| 2017 Aug. 6 | 7196 |
| 2017 Aug. 7 | 7113 |
| 2017 Aug. 8 | 7064 |
| 2017 Aug. 9 | 6966 |
| 2017 Aug. 10 | 6967 |

We use the NMSE to assess the performance of the model. There is the definition of Normalized Mean Squared Errors (NMSE).

$$NSME = {\sum }_{i} \frac{{( {P}_{i} - {M}_{i} )}^{2}}{\overline{P} \overline{M}}$$

$$\overline{P} = \frac{1}{N} {\sum }_{i} {P}_{i}$$

$$\overline{M} = \frac{1}{N} {\sum }_{i} {M}_{i}$$

With the formula provided above, the FitBliss platform obtains the result of NMSE and accuracy.

Accuracy=1−Average of Error Rate=1−Sum of Error Rate/7

Error Rate=the difference between predicted values and real values/real values

For example:

For the Error Rate in 2017-08-04, Error Rate=(7748−7527)/7527=0.0296

For the Error Rate in 2017-08-05, Error Rate=(7427−7397)/7397=0.0041

Based on the formula provided above, the model achieves an accuracy of 0.986399.

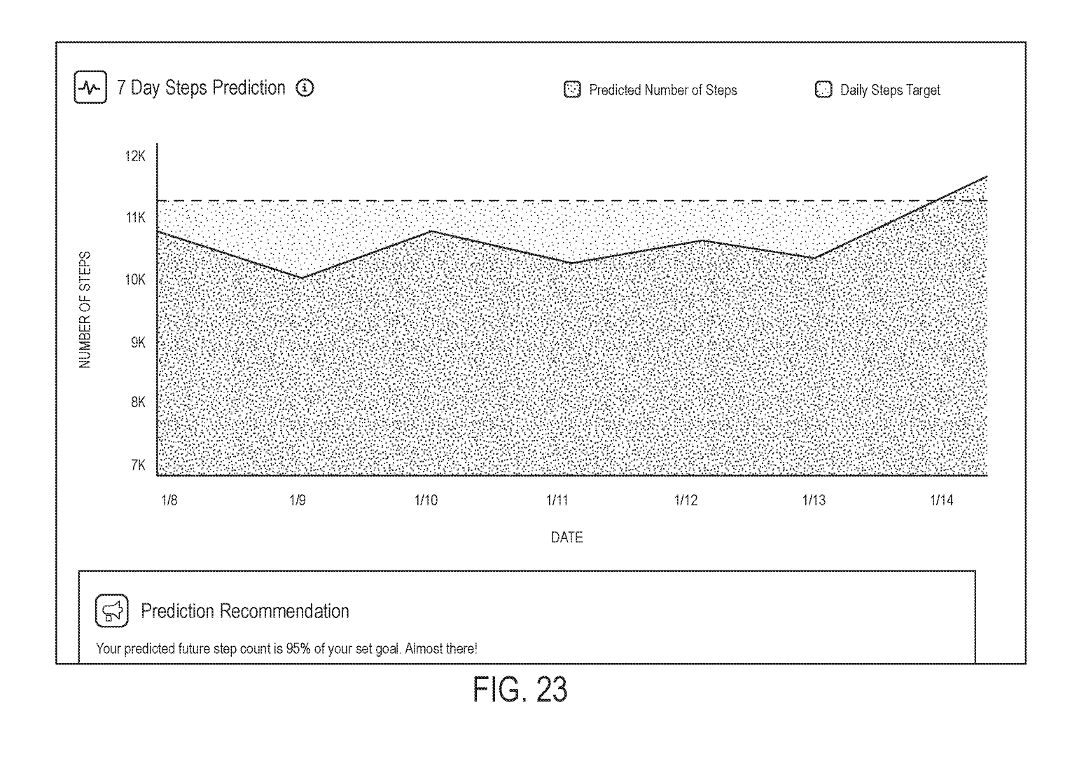

FIG. 23 shows an example of plots for the predicted steps trend in next 7 days and daily steps target. In addition, we give the distance between predicted steps and steps target by quantifying the percentage of average future steps to the set goal. The section “Prediction Recommendation” of FIG. 23 shows that “Your predicted future step count is 95% of your set goal”. The figure gives users a full view of their own physical activity intensity in the next weekly plan, propelling users to adjust exercise strategies accordingly.

### 4. Health Tips & Digital Content Recommendation System

Under an embodiment FitAssistant provides users with personalized suggestions based on the studied exercise patterns and steps intensity prediction. Health Tips are called FitTips inside FitBliss, and FitVideos are health videos generated by FitBliss either through FitBliss owned content and/or through a partnership with a content database.

FitAssistant currently comprises 1,000 FitTips & FitVideos (Health Tips) in a health content database in total. These tips/videos are presented as a Health Tips Recommendation System in FitAssistant. The tips/videos are categorized by the studied exercise patterns and steps intensity predictions.

An embodiment of FitAssistant may rotate Health Tips as follows.

Health Tips are divided into 4 categories by the time of the day. They are:

| | | |
|---|---|---|
| 1. | Early Morning | 8:00 am-9:59 am |
| 2. | Late Morning | 10:00 am-12:00 pm |
| 3. | Afternoon | 12:00 pm-7:00 pm |
| 4. | Evening | 7:00 pm-10:00 pm |

Health Tips used in FitAssistant are classified into two categories according to the predictions on the steps trend of users which reflects the intensity of their physical activity.

Condition 1: The average of next 7 days steps trend is equal or greater than steps goal.

Condition 2: The average of next 7 days steps trend is less than steps goal.

Condition 1 indicates that the user group usually has regular exercise habits and the steps goal is achievable in their current routine. FitAssistant tailors under an embodiment the FitTips that are suitable for customers with high intensity exercise training, including how to estimate the max limit of physical activity intensity, how to relax muscles after workout and how to plan HIIT (High Intensity Interval Training), etc.

Condition 2 indicates that most in the user group are beginners and there might be obstacles in achieving the recommended steps goal despite determination for personal health management. FitAssistant tailors under an embodiment the FitTips that are suitable for customers with relatively low exercise training, including how to warm up before exercise, how to split their workout time into small slots within one day, etc.

FitTips comprises a personalized health tips & digital content recommendation system, which directly contributes to the number of steps and the intensity of physical activity Below is an example of FitTips recommended for users whose next 7 day step trend average is equal to or greater than steps goal from health content database. The tables below illustrates the Tip # in the database of Health FitTips and FitVideos.

| Time Slot | Exercise (125 number of tips in every time slot) |
|---|---|
| Early Morning | 63, 64, 67, 195, 201, 202, 203 |
| Late Morning | 69, 72, 185, 186, 204, 205, 206 |
| Afternoon | 78, 192, 193, 194, 196, 199, 200 |
| Evening | 81, 84, 85, 103, 181, 197, 198 |

Below is an example of FitTips recommended for users whose next 7 day step trend average is less than steps goal from health content database.

| Time Slot | Exercise (125 tips in every time slot) |
|---|---|
| Early Morning | 27, 28, 65, 66, 63, 183, 184 |
| Late Morning | 69, 70, 72, 73, 186, 187, 188 |
| Afternoon | 74, 75, 76, 77, 136, 29, 189 |
| Evening | 30, 82, 83, 85, 180, 190, 191 |

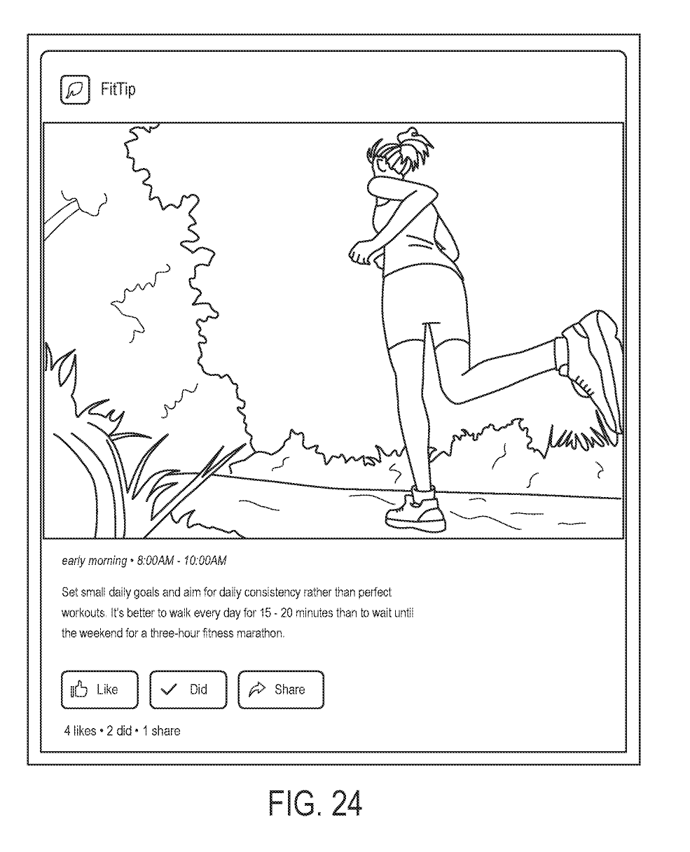

FIG. 24 show a screen shot of FitTips under an embodiment of FitAssistant. FIG. 24 shows content description with image, FitTip time slot, buttons for tracking customer feedback, real-time statistics of FitTips engagement, and the flexibility to share among users groups.

Under an embodiment, machine learning my assume the task of health content classification, thereby avoiding the large amount of time spent on content classification by manual work and increasing the metrics to tailor the personalized health content.

### Data Architecture

Taking the mission of optimizing health and productivity for a global workforce as a priority, an embodiment of FitAssistant integrates the AI FitAssistant with CRM (Customer Relationship Management) platforms. Such platforms include Salesforce™ along with and not limited to Sage™, Workday Inc.™ and Ultimate Software™ as well. FitAssistant integrates with Slack™ and Teams™ (by Microsoft™).

The following disclosure describes the data architecture of FitAssistant which integrates Salesforce and AWS, a cloud computing system, under an embodiment.

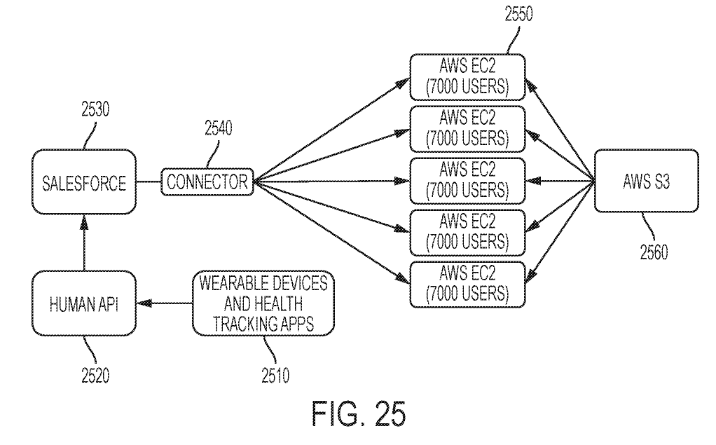

FIG. 25 shows a process of data collection and integration under an embodiment.

### Data Collection and Integration

FIG. 25 shows data query from wearable devices and health tracking apps. (2510) e.g.: steps, distance, sleep, calories burned, exercise minutes, sedentary minutes, heart rate, nutrition/diet, glucose, floors, types of activities, etc. Data is integrated from different resources (e.g.: apple watch, Titbit) into Human API 2520. Note that Human API comprises a software integration solution vendor that provides api access to all the devices and wearables integrated into the FitBliss platform.

### Data Transfer and Storage

Data is transferred from Human API to Salesforce 2530 to be used in product development. Data is stored under one embodiment in an object. Date, Steps, and Usernames are stored under corresponding fields in an embodiment.

Note that connector 2540 works as a driver to transfer data between salesforce and AWS in real time bidirectionally.

Data is transferred from Salesforce to AWS EC2 (or analogous cloud computing architecture) in preparation for training prediction models in AWS EC2. The procedures and properties of AWS EC2 are described as follows:

The health tracking data are queried through the Salesforce.com Rest API client as the connector mentioned above. The package “Salesforce” in python provides an interface to the REST resource and APEX API by enabling SOQL (Salesforce Object Query Language) query in python, returning a dictionary of the API JSON response.

Data corresponding to the salesforce of multiple organizations may be forwarded to respective AWS EC2 virtual machines. FIG. 25 shows five virtual machines 2550 each capable of processing data for 7000 users of respective organizations.

### Training Prediction Model

The prediction model is trained on AWS EC2. As mentioned above, there are five virtual machines running at the same time considering both efficiency and price to support data processing for multiple users, under an embodiment. FIG. 25 shows that the max number of active user data that each VM supports is 7000, under an embodiment. Hence, the max capacity of active users' data for these five virtual machines is 35,000.

### Data Transfer and Storage

After the predictions are done, the prediction results and steps trend for each individual is sent in two directions: 1. Salesforce database; 2. Data backup.

Under one embodiment, prediction results and step trend data are sent to Salesforce. The systems and methods of an embodiment use the Bulk API, an optimal Restful API for transferring large datasets, to upload prediction results and steps trend into database of Salesforce. Data are stored in a Steps Predicted field and Steps Moving Avg field under a FitBliss object. On the other hand, the prediction results and steps trend are sent to backup.

FitAssistant may then implement data visualization in Salesforce, under an embodiment. The prediction results and steps trend may be displayed in respective charts “Virtual's Next 7 Days Steps Trend and Goal” and “Virtual's Steps Trend and Goal”, reflecting the intensity trend of one's physical activity and future steps trend.

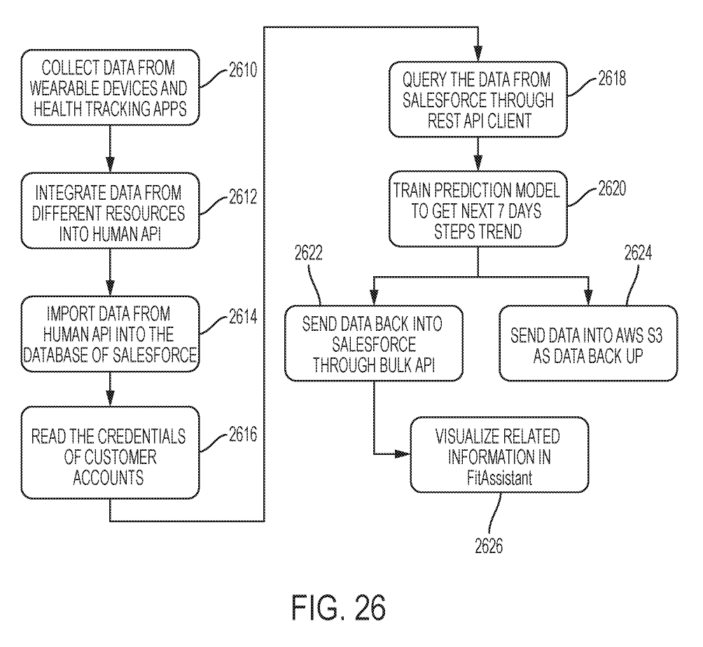

FIG. 26 shows steps of data collection and integration, under an embodiment. FIG. 26 shows the step 2610 of collecting data from wearable devices and health tracking applications. FIG. 26 shows the step 2612 of integrating data from different resources into Human API. FIG. 26 shows the step of 2614 importing data from Human API into the database of Salesforce. FIG. 26 shows the step of 2616 reading the credentials of customer's accounts. FIG. 26 shows the step 2618 of querying data from Salesforce through a REST API client. FIG. 26 shows the step 2620 of training a prediction model to get the next seven day trend. FIG. 26 shows the step 2622 of sending data back to Salesforce through BULK API and the step 2624 of sending data to back up. FIG. 26 shows the step 2626 of visualizing model output through FitAssistant.

Under an embodiment, a FitBliss Tab comprises the homepage of the FitBliss application. It may provide a navigation bar for user experience. It may highlight a calendar of days employee had logged an activity (either from Routine Logs and/or FitConnect Activities as further described below). It may highlight weekly exercise minutes or a duration bar of chart or graph for immediate real-time performance tracking. The tab may highlight last logged mood (from either Routine Log/Fitconnect Activity). The Tab may also comprise embedded Chatter feed for immediate social experience. The Tab may also include a list of last modified 5 Favorite FitRoutines for easy navigation to log workout (through FitRoutines as further described below). The Tab may also include a Wellness Topic of the month that comes from FitBliss via webservice call.

The FitBliss platform provides one or more interfaces for receiving and providing input from users. The interfaces are referred to as FitRoutines, Routine Log, Nearby Activities, Daily Summary, FitConnect Activities, and FitChallenge. Each interface corresponds to data objects and/or object structures for receiving, managing, and presenting user data. Additional objects may include FitConnect and App Connect™ as further described below.

A user of the FitBliss platform may use a FitRoutines interface to create their own form of a routine that is personal to their lifestyle (morning run, tennis, CrossFit, bootcamp, and more). The FitRoutines interface may receive the following data: Title (of the FitRoutine); Description (of the FitRoutine); Favorite (Routine most likely to repeat often).

A user of the FitBliss platform may use the Routine Log interface to keep track of routines. The interface facilitates logging fitroutines created by users. The Routing Log interface may receive the following data: Name of Log, Geo-Location (via Google Maps™), Partners (colleagues or non-colleagues), Duration, Mood, Log Date & Time.

A user of the FitBliss platform may use a Nearby Activities interface to view/monitor locations of both Routine Logs & FitConnect Activities. The interface may comprise a map of every workout logged nearby. Filters may be provided including My Routines (i.e., routines of a user), Follow (i.e., routines of those individuals followed by user), Time Interval filters (i.e., this week, this month), Mood filter (see Routine Log interface described above). Under one embodiment a user may only see the data of other users followed in FitBliss. Users may click on activity icons to see details of an associated Routine Log or activity created by the corresponding user (see the FitConnect Activity interface described below).

A FitConnects object pulls in the data from wearables/applications from the user syncing via App Connect™ (an integration portal). Such data may include Steps, Distance, Sedentary Minutes, Calories, Activity Minutes (minutes logged from apps), Floors, and List of Activities being pulled from App Connect™ with information of the activity. FitConnect is not shown to the users but is available to the administrators of the instance of FitBliss per customer.

A user of the FitBliss platform may use App Connect™ to synchronize the FitBliss platform with their applications and devices. App Connect™ provides selectable interface buttons corresponding to data sources, e.g. Fitbit™ or Apple Watch™, where users can sync their devices/applications. App Connect™ comprises an integration layer between the FitBliss Platform and the wearables/fitness applications.

A Daily Summary interface may provide summary data of each day. The summary information may include Steps (via App Connect™), Distance (via App Connect™), Sedentary Minutes (via App Connect™), Calories (via App Connect™), Activity Minutes (minutes logged from apps & routine logs), Floors (via App Connect™), Routine Logs (via FitRoutine Object, Routine Logs), Commute Button for Walking/Biking to & from work (Updated from the Daily Summary Page), Personal Note of the Day—users provide a description of what motivated them to exercise, their weight, and any other notes that's personal to users, Featured Photo of the Day—users may upload pictures from their phone or computer that highlights the day.

A FitConnect Activities Object pulls/organizes logged activity data using data obtained through synchronization of FitBliss with applications/wearables. The FitConnect Activities Object may include Name, Source, Date, Start & End Time, Steps (if provided), Minutes of Activities, How Do You Feel?, FitPartner Member, Calories (if provided), Distance (if provided), Location, and Commute (indicating that the user is walking or biking to and from work instead of driving or taking the bus for instance). The FitBliss platform may present this data to the user for each logged activity under an embodiment.

A user of the FitBliss platform may use the FitChallenge interface to self-create a steps challenge. A first user may create the challenge, create teams, duration of challenge, and then add other users to each team. A corresponding object may comprise Challenge Name, Team Names, and Team Members. The Steps Data may be received from any device that is synced by a user from the App Connect™ object to the Daily Summary or the FitConnect Activity object.

A method of an embodiment is described herein that comprises receiving from the user a selection of a fitness activity. The method includes receiving from the user a productivity goal for achieving at least one productivity objective, the receiving including indicating a time frame for achieving the at least one productivity goal. The method includes using historical data of the user to identify and recommend a fitness goal, wherein the historical data includes fitness activity data of the fitness activity previously performed by the user and productivity data of productivity objectives previously achieved by the user over a common time frame, wherein the productivity objectives include the at least one productivity objective. The method includes correlating the fitness activity data and the productivity data to identify an interdependence between the fitness activity data and the productivity data, the using the historical data of the user comprising using information of the interdependence to identify and recommend the fitness goal, wherein the fitness goal comprises achieving a target level of the fitness activity within the time frame. The method includes instructing the user to achieve the fitness goal.

Computer networks suitable for use with the embodiments described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network may be any microprocessor controlled device that permits access to the network, including terminal devices, such as personal computers, workstations, servers, mini computers, main-frame computers, laptop computers, mobile computers, palm top computers, hand held computers, mobile phones, TV set-top boxes, or combinations thereof. The computer network may include one of more LANs, WANs, Internets, and computers. The computers may serve as servers, clients, or a combination thereof.

The systems and methods for tracking and exchanging wellness, fitness, and productivity data using an electronic platform can be a component of a single system, multiple systems, and/or geographically separate systems. The systems and methods for tracking and exchanging wellness, fitness, and productivity data using an electronic platform can also be a subcomponent or subsystem of a single system, multiple systems, and/or geographically separate systems. The components of systems and methods for tracking and exchanging wellness, fitness, and productivity data using an electronic platform can be coupled to one or more other components (not shown) of a host system or a system coupled to the host system.

One or more components of the systems and methods for tracking and exchanging wellness, fitness, and productivity data using an electronic platform and/or a corresponding interface, system or application to which the systems and methods for tracking and exchanging wellness, fitness, and productivity data using an electronic platform is coupled or connected includes and/or runs under and/or in association with a processing system. The processing system includes any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein refers to any logic processing unit, such as one or more central processing units (CPUs), digital signal processors (DSPs), application-specific integrated circuits (ASIC), etc. The processor and memory can be monolithically integrated onto a single chip, distributed among a number of chips or components, and/or provided by some combination of algorithms. The methods described herein can be implemented in one or more of software algorithm(s), programs, firmware, hardware, components, circuitry, in any combination.

The components of any system that include the systems and methods for tracking and exchanging wellness, fitness, and productivity data using an electronic platform can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the systems and methods for tracking and exchanging wellness, fitness, and productivity data using an electronic platform and corresponding systems and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the systems and methods for tracking and exchanging wellness, fitness, and productivity data using an electronic platform and corresponding systems and methods include: microcontrollers with memory (such as electronically erasable programmable read only memory (EEPROM)), embedded microprocessors, firmware, software, etc. Furthermore, aspects of the systems and methods for tracking and exchanging wellness, fitness, and productivity data using an electronic platform and corresponding systems and methods may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein may be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the above described components may be processed by a processing entity (e.g., one or more processors) within the computer system in conjunction with execution of one or more other computer programs.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import, when used in this application, refer to this application as a whole and not to any particular portions of this application. When the word “or” is used in reference to a list of two or more items, that word covers all of the following interpretations of the word: any of the items in the list, all of the items in the list and any combination of the items in the list.

The above description of embodiments of the systems and methods for tracking and exchanging wellness, fitness, and productivity data using an electronic platform is not intended to be exhaustive or to limit the systems and methods to the precise forms disclosed. While specific embodiments of, and examples for, the systems and methods for tracking and exchanging wellness, fitness, and productivity data using an electronic platform and corresponding systems and methods are described herein for illustrative purposes, various equivalent modifications are possible within the scope of the systems and methods, as those skilled in the relevant art will recognize. The teachings of the systems and methods for tracking and exchanging wellness, fitness, and productivity data using an electronic platform and corresponding systems and methods provided herein can be applied to other systems and methods, not only for the systems and methods described above.

The elements and acts of the various embodiments described above can be combined to provide further embodiments. These and other changes can be made to the systems and methods for tracking and exchanging wellness, fitness, and productivity data using an electronic platform and corresponding systems and methods in light of the above detailed description.

I claim:

1. A method comprising,
receiving from the user a selection of a fitness activity;
receiving from the user a productivity goal for achieving at least one productivity objective, the receiving including indicating a time frame for achieving the at least one productivity goal;
using historical data of the user to identify and recommend a fitness goal, wherein the historical data includes fitness activity data of the fitness activity previously performed by the user and productivity data of productivity objectives previously achieved by the user over a common time frame, wherein the productivity objectives include the at least one productivity objective;
correlating the fitness activity data and the productivity data to identify an interdependence between the fitness activity data and the productivity data, the using the historical data of the user comprising using information of the interdependence to identify and recommend the fitness goal, wherein the fitness goal comprises achieving a target level of the fitness activity within the time frame; and
instructing the user to achieve the fitness goal.
